(19) United States
(12) Patent Application Publication
(10) Pub. No.: US 2026/0243753 A1
(43) Pub. Date: Aug. 20, 2026

(54) BODY FLUID ANALYSIS METHOD AND SYSTEM

(71) Applicant: Hitachi High-Tech Solutions Corporation, Tokyo (JP)

(72) Inventors: Takeshi AISO, Tokyo (JP); Tsukasa SHISHIKA, Tokyo (JP); Hiroaki HASHIMOTO, Tokyo (JP); Shoichi UECHI, Tokyo (JP)

(21) Appl. No.: 19/159,415

(22) PCT Filed: Jan. 17, 2024
(86) PCT No.: PCT/JP2024/001132

§ 371 Date: Aug. 25, 2025

(30) Foreign Application Priority Data
Aug. 23, 2023 (JP) 2023-135737

(51) Int. Cl. G01N 33/487

(52) U.S. Cl. CPC G01N 33/487

(57) ABSTRACT

The possibility of intentional switching or falsification of a sample or unintentional mix-up or misidentification thereof is determined/detected/output. A body fluid analysis method (test method) uses a mass spectrometer as a test apparatus to analyze a body fluid, which is a sample collected from a test subject.

The mass spectrometer has a function of introducing the sample and measuring and analyzing a component amount of a target component substance on the basis of mass spectrometry. The body fluid analysis method includes a determination step of determining, as a result of the measurement of the sample by the mass spectrometer, a possibility that there has been switching or mix-up of the sample with an inappropriate sample on the basis of the component amount of the target component substance and an output step of outputting a result of the determination when the sample is determined to be inappropriate.

### TECHNICAL FIELD

The present disclosure relates to technology for body fluid analysis and drug testing.

### BACKGROUND ART

Various drug tests, i.e., body fluid analyses have been performed. For example, there are drug tests for entrants upon entry, alcohol and drug tests for drivers, and doping tests for athletes. In a doping test or the like, a detailed examination may also be performed using urine and blood as samples. Examples of target drugs, i.e., target component substances include illegal drugs such as narcotics, prohibited substances as defined in competitions, and the like. A detailed examination takes a relatively long time to produce a result. Accordingly, there has been an increasing demand for a simple test that takes a relatively short time, i.e., a screening test.

For the various drug tests/body fluid analyses, a mass spectrometer can be used as a drug test apparatus, i.e., a body fluid analyzer. During a drug test/body fluid analysis, e.g., a skin tissue is wiped as a body fluid from skin of a test subject to be used as a sample. In other words, the sample is a specimen, a sample, a test subject, or the like.

The body fluid also includes sweat and saliva. The mass spectrometer measures/analyzes, for a sample that has been set and introduced, component substances on the basis of known mass spectrometry. From a result of the measurement/analysis, it can be detected/estimated whether or not a body fluid, which is the sample, includes a target substance such as, e.g., an illegal drug.

Mass spectrometry technology (Mass Spectrometry) is a technology of ionizing a sample on atomic/molecular levels, measuring and analyzing a mass, and identifying and quantifying a substance. The mass spectrometer ionizes a sample in vacuum under a high voltage, separates ions flying with electrostatic power by using an electromagnetic action according to a mass-to-charge ratio thereof, and detects separated components. By such measurement, a mass spectrum (Mass Spectrum, which may be referred to also as Mass) is obtained. In the mass spectrum, an abscissa axis represents the mass-to-charge ratio (m/z, where m is a mass and z is a charge), while an ordinate axis represents a signal intensity.

Examples of the mass spectrometer capable of body fluid analysis, i.e., the test apparatus include Japanese Patent No. 6368067 (Patent Literature 1). Patent Literature 1 describes detection of whether a sample is a substance among body liquid components including at least one of urine, sweat, and saliva or a solid substance or the like.

Meanwhile, Japanese Patent No. 7002637 (Patent Literature 2) describes an example of a blood test support system and describes management of the date and time of self blood

### CITATION LIST

### Patent Literature

Patent Literature 1: Japan Patent No. 6368067
Patent Literature 2: Japan Patent No. 7002637

### SUMMARY OF INVENTION

### Technical Problem

A sample to be tested/analyzed in a body fluid analyzer, which is a test apparatus, may be subject to intentional switching or falsification, unintentional mix-up or misidentification, or the like. In other words, there are cases where a sample introduced in the test apparatus is not from the body fluid of a test subject of concern, but is from a substance other than that from the person of concern. In that case, the test apparatus cannot perform appropriate testing/analysis. In a system including the test apparatus, it is desirable to determine/detect/output that the sample of concern may possibly be a sample from a person other than the person of concern, i.e., that there is a possibility of intentional switching or the like of the sample, unintentional mix-up or the like thereof, or the like. In other words, it is desirable that the system including the test apparatus has a function of being capable of determining/detecting/outputting a possibility as described above as indicating that the sample is not appropriate, i.e., the sample is inappropriate. A related-art example does not have such a function.

In a case where, at the time of testing, a person confirming the test, i.e., person providing the test collects a sample directly from a test subject, i.e., measurement subject, and immediately provides the test using the test apparatus on the spot, the possibility of the foregoing switching is low, and therefore a function as described above is not necessarily required.

Meanwhile, in a case where a location where the sample is collected from the test subject is different from the location where the test is provided, there is a possibility of the foregoing switching or the like, and therefore a function as described above is required and effective. For example, a sample is collected from the test subject at the location such as an airport, a port, a checkpoint, or an athletic venue, and the collected sample is transported by a means such as mailing another location where the test apparatus is located. Then, the samples is introduced into the test apparatus at that location, and the test is provided. Such a test may be referred to also as off-site monitoring. In this case, a test provider who provides the test at the location of the test apparatus is not face-to-face with the test subject, and it is uncertain whether the transported sample is from a body fluid collected appropriately from the test subject of concern.

Examples of the switching or falsification of the sample include a case where a substance is collected from another person, an animal, or any other object other than the test subject of concern, such as a wall, a desk, a bag, clothing, food, or a drink. Examples of sample mix-ups and misidentifications include cases where samples are collected appropriately, but a plurality of samples from a plurality of test subjects are mistakenly switched in the process of collecting work, mailing, or the like.

When there is any switching of the foregoing samples or the like, as long as it is possible to determine/detect/output a possibility that the sample is from someone other than the person of concern or the sample is an inappropriate, it is possible to take measures such as reconfirming the sample set in the test apparatus, re-collecting and re-testing of the sample, or temporary suspension of a testing operation. Consequently, this contributes to optimized or efficient drug testing/body fluid analysis.

An object of the present disclosure is to provide a technology that allows a possibility of intentional switching or falsification of samples, unintentional mix-ups or misidentifications thereof, or the like to be determined/detected/output in relation to the body fluid analysis and drug testing technology mentioned above.

### Solution to Problem

A representative embodiment of the present disclosure has a configuration shown below. The embodiment is the body fluid analysis method of using a mass spectrometer serving as a test apparatus to analyze the body fluid, which is a sample collected from a test subject, the mass spectrometer being an apparatus having a function of introducing the sample and measuring and analyzing a component amount of a target component substance on the basis of mass spectrometry, the body fluid analysis method including, as steps to be performed by the mass spectrometer: a determination step of determining, as a result of the measurement of the sample by the mass spectrometer, a possibility of switching or mix-up of the sample to be an inappropriate sample on the basis of the component amount of the target component substance; and an output step of outputting a result of the determination when the sample is determined to be inappropriate.

### Advantageous Effects of Invention

According to the representative embodiment of the present disclosure, it is possible to determine/detect/output the possibility of intentional switching or falsification of samples, unintentional mix-ups or misidentifications thereof, or the like to be determined/detected/output in relation to the body fluid analysis and drug testing technology mentioned above. Problems, configurations, effects, and the like other than those described above will be described in a mode for implementing the invention.

### BRIEF DESCRIPTION OF DRAWINGS

FIG. 1 is a diagram illustrating an example of an overall configuration of a test system that implements a test method in a first embodiment.

FIG. 2 is a sequence diagram of a simple drug test service in a test method/test system in the first embodiment.

FIG. 3 is a diagram illustrating an outline of the simple drug test service in the test method/test system in the first embodiment.

FIG. 4 are diagrams each illustrating an example of an outer appearance configuration of a mass spectrometer as a test apparatus in the test method/test system in the first embodiment.

FIG. 5 is a diagram illustrating a functional block configuration of the mass spectrometer as the test apparatus in the test method/test system in the first embodiment.

FIG. 6 is a diagram illustrating a configuration of a mass measurement unit and the like of the mass spectrometer in the test method/test system in the first embodiment.

FIG. 7 are diagrams each illustrating an example of a mass spectrum obtained by the mass spectrometer in the test method/test system in the first embodiment.

FIG. 8 is a diagram illustrating an example of a configuration of a computer system included in a PC of the test apparatus in the test method/test system in the first embodiment.

FIG. 9 is a diagram illustrating an example of a configuration of a computer system included in a server in the test method/test system in the first embodiment.

FIG. 10 is a diagram illustrating a flow of processing of testing a sample in the test apparatus in the test method/test system in the first embodiment.

FIG. 11 is a diagram illustrating an example of a configuration of a questionnaire form in the test method/test system in the first embodiment.

FIG. 12 is a diagram illustrating an example of a configuration of a questionnaire input screen in the test method/test system in the first embodiment.

FIG. 13 is a diagram illustrating an example of a configuration of a screen for inputting test subject information in the test method/test system in the first embodiment.

FIG. 14 is a diagram illustrating an example of a configuration of a report form for notification of a test result in the test method/test system in the first embodiment.

FIG. 15 is a diagram illustrating an example of the configuration of the report form for the notification of the test result in the test method/test system in the first embodiment.

FIG. 16 is diagram illustrating an example of a customer ID management table in the test method/test system in the first embodiment.

FIG. 17 is an illustrative view of a function of determining an inappropriate sample in the PC of the test apparatus in the test method/test system in the first embodiment.

FIG. 18 is an illustrative view of details of the function of determining the inappropriate sample in the test method/test system in the first embodiment.

FIG. 19 are diagrams each illustrating an example of a m/z signal intensity of a mass spectrum as an example of a component amount of a target component substance in the test method/test system in the first embodiment.

FIG. 20 is a diagram illustrating an example of the mass spectrum when impurities are contained in large amounts in the test method/test system in the first embodiment.

FIG. 21 is a diagram illustrating an example of processing of a determination condition 1 in the test method/test system in the first embodiment.

FIG. 22 is a diagram illustrating an example of processing of a determination condition 2 in the test method/test system in the first embodiment.

FIG. 23 is a diagram illustrating an example of processing of a determination condition 3 in the test method/test system in the first embodiment.

FIG. 24 is a diagram illustrating an example of a component amount and a reference range in the test method/test system in the first embodiment.

FIG. 25 is a diagram illustrating an example of a screen displayed on the PC of the test apparatus in the test method/test system in a modification of the first embodiment.

FIG. 26 is a diagram illustrating an example of generation of the reference range or the like in the test method/test system in the modification of the first embodiment.

### DESCRIPTION OF EMBODIMENTS

Hereinafter, one embodiment of the present disclosure will be described with reference to the accompanying drawings. In the drawings, the same portions are denoted by the same reference signs in principle to omit a repetitive description. In the drawings, for easier understanding of the invention, representations of components may not show actual positions, sizes, shapes, ranges, and the like thereof.

For the purpose of explanation, when describing processing by a program, the program, function, processing unit, or the like may be described as the main body, but the main body as hardware therefor is a processor or a controller, an apparatus, a calculator, a system, or the like including the processor or the like. The calculator causes the processor to perform processing according to a program read into a memory, while using resources such as the memory and a communication interface. Consequently, a predetermined function, processing unit, or the like is implemented. The processor is configured to include a semiconductor device such as, e.g., a CPU/MPU, a GPU, or the like. Processing is not limited to software program processing, and can also be performed by a dedicated circuit. To the dedicated circuit, a FPGA, an ASIC, a CPLD, or the like is applicable.

The program may be installed in advance as data in a target calculator, or may also be distributed as data from a program source to the target computer. The program source may be a program distribution server in a communication network, or may also be a non-transitory computer readable storage medium, e.g., a memory card or disk. The program may also be configured to include a plurality of modules. A computer system may also be configured to include a plurality of apparatuses. The computer system may also be configured to include a client-server system, a cloud computing system, an IoT system, and the like. Various data and information is configured to include structures such as, e.g., a table and a list, but is not limited thereto. Expressions such as identification information, identifiers, IDs, names, and numbers are replaceable with each other.

### First Embodiment

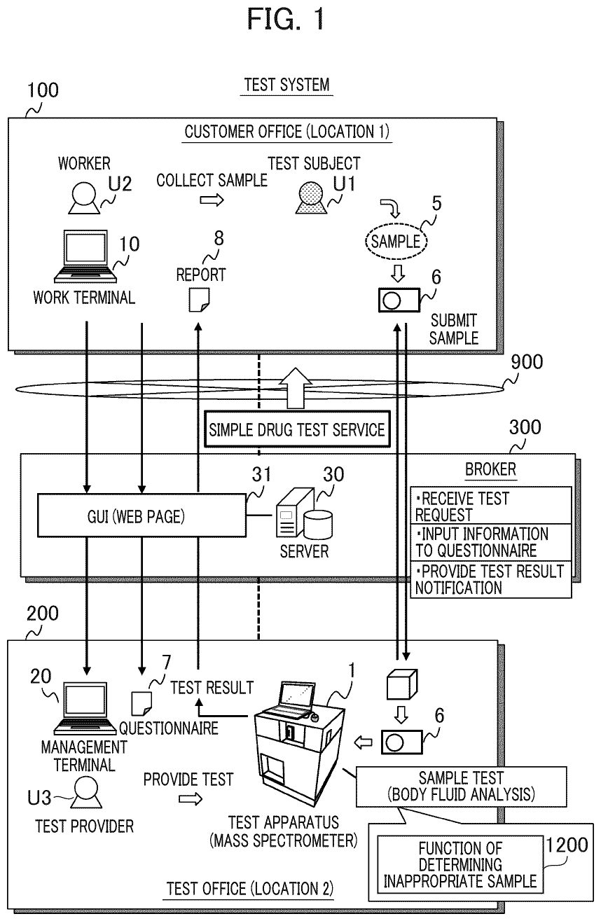

Referring to FIG. 1 and subsequent drawings, a description will be given of a test method and a test system according to the first embodiment of the present disclosure.

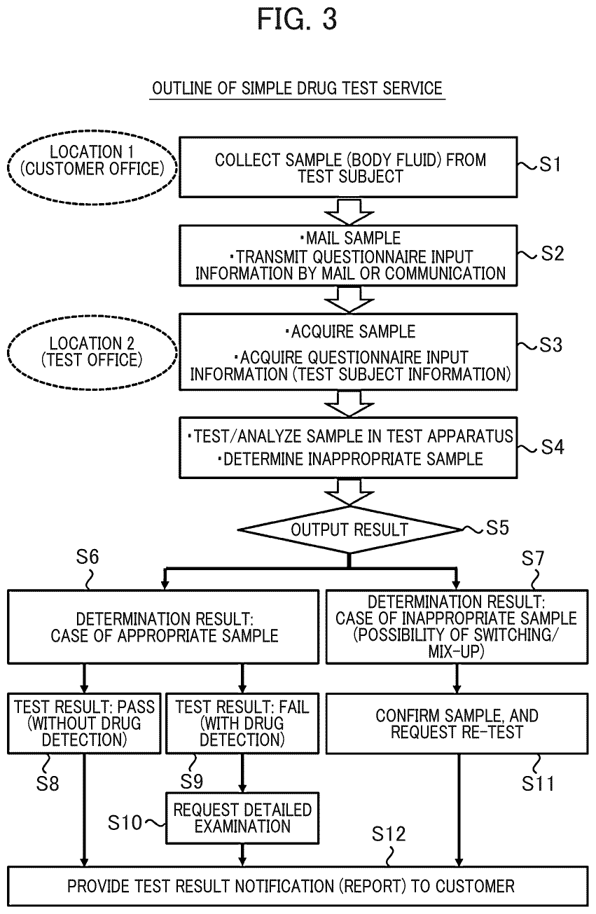

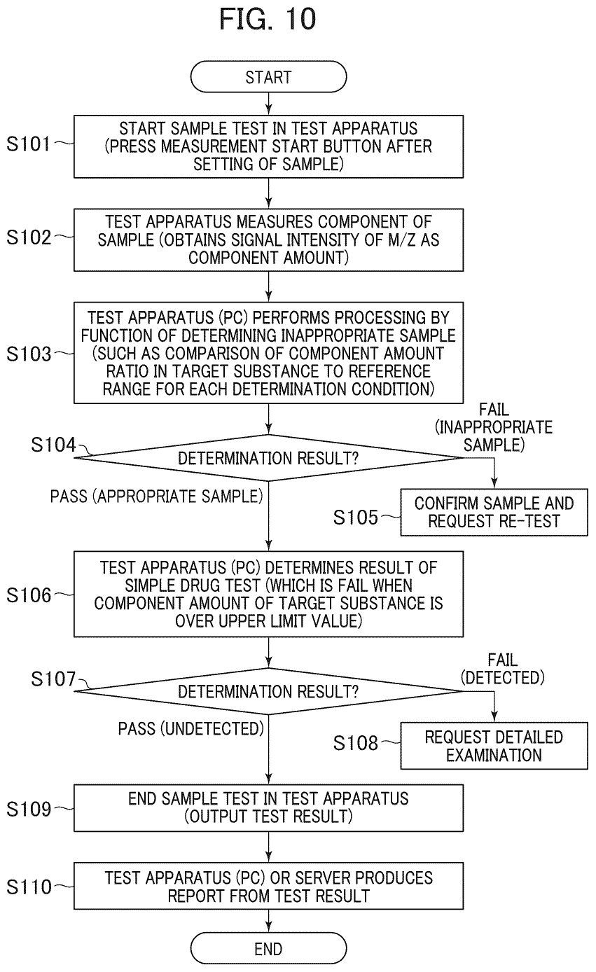

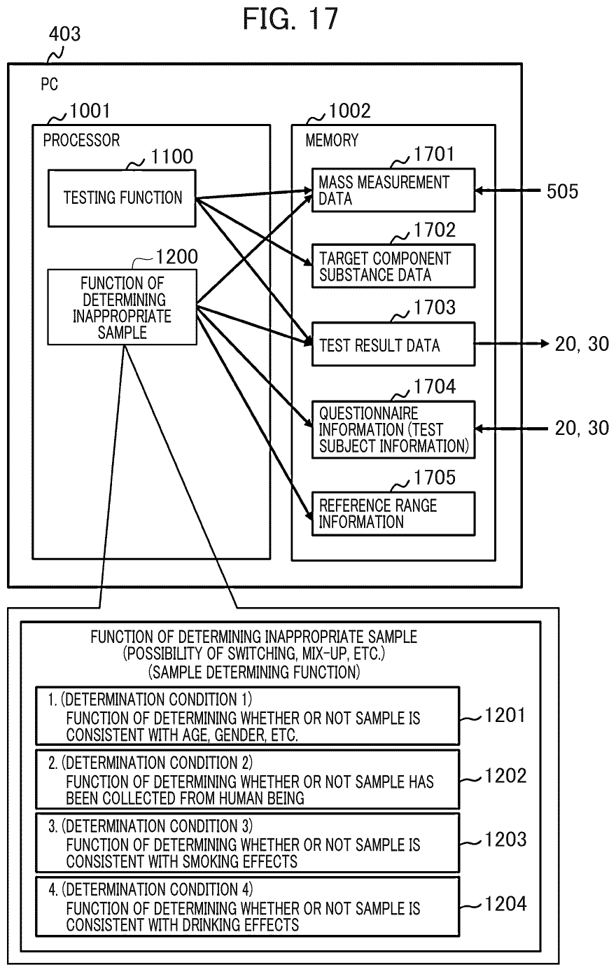

In other words, the test method in the first embodiment is a body fluid analysis method. The test method in the first embodiment is a method including steps to be performed in the test system (i.e., the body fluid analysis system) in the first embodiment. As illustrated in FIG. 1, FIG. 3, FIG. 10, FIG. 17, and the like, the test method in the first embodiment has, as a characteristic function, a function 1200 of determining/detecting/outputting whether or not a sample is an inappropriate sample which has not been collected from a person of concern in a test in the test apparatus 1.

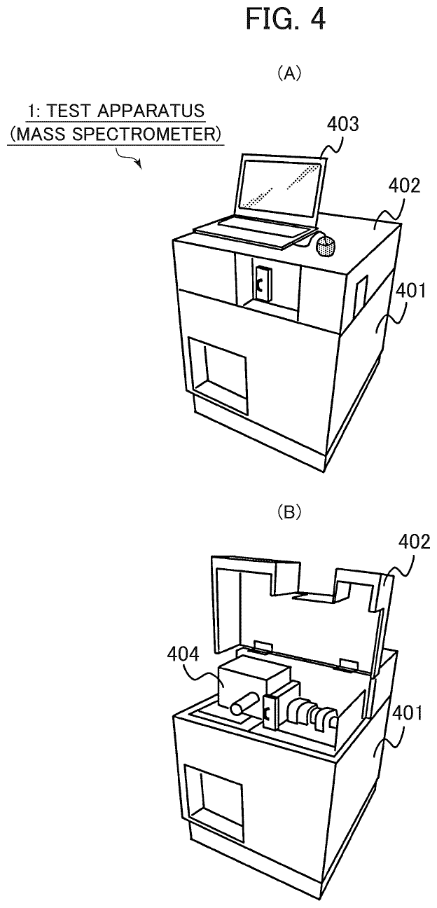

It is assumed that, in a mode taken by the first embodiment, processing for the characteristic function is mainly performed by a PC 403 (FIG. 4) of the test apparatus 1, but is not limited thereto. For example, it may also be possible that test result data in the test apparatus 1 or the like is acquired by another computer, e.g., a server 30, a management terminal 20, or the like (FIG. 1), and the processing for the characteristic function is performed by the server 30, the management terminal 20, or the like. Alternatively, the processing may also be implemented by being shared/coordinated among a plurality of computers such as the PC 403 and the server 30.

In the first embodiment, a simple drug test/screening test in which a location (assumed to be a location 1) where a sample is collected is different from a location (assumed to be a location 2) where the test is provided is provided as service to a customer by mail and communication (FIG. 1). For example, on the test subject such as an entrant, a driver, an athlete, or an employee, the simple test/screening test related to illegal drugs or the like is performed. The test contributes to ensuring work safety, health management, or the like. The test can be provided as, e.g., a pre-employment test, a periodical test, a post-accident test, or the like with predetermined timing according to a customer to be tested.

### Test System

FIG. 1 illustrates an overall configuration of a system including the test system (body fluid analysis system) in the first embodiment. The system in FIG. 1 is referred to also as the test system. The test system in FIG. 1 is a system that provides simple drug test service to a customer office 100 for customers. The test system in FIG. 1 is a system in which a system of the customer office 100, a system of a broker 300, and a system of a test office 200 of a service provider who provides a test are connected to each other via a communication network 900. Note that there may also be a plurality of the customer offices 100 for customers who receive the service. The communication network 900 includes the Internet or the like, and includes a mailing system or the like.

The customer office 100 is at the location 1 serving as a given location, while the test office 200 is at the location 2 serving as another location. The customer office 100 has one or more test subjects U1 who receive the test. At the customer office 100, a worker U2 for the customers performs work of collecting a sample 5 from the test subject U1. In a specific example, the worker U2 wipes off a skin tissue from neck skin of the test subject U1 with gauze and stores the gauze in a sample holder 6 (or in a plastic bag). The sample holder 6 may be a dedicated container that can be set in the test apparatus 1, or may also be another container, e.g., a plastic bag. The worker U2 can operate a work terminal 10 such as a PC in the customer system and access a Web page 31 of the broker 300. The worker U2 can perform requesting of the test, inputting of a questionnaire 7, viewing of a report 8 of a test result, and the like on the Web page 31 from the work terminal 10.

At the location 2 of the test office 200, the test apparatus 1 is placed. To the location 2 of the test office 200, the sample holder 6 with the sample 5 is sent by mail from the customer office 100. A test provider U3 at the test office 200 uses the test apparatus 1 to perform work of testing the sample 5. When the sample holder 6 in which the sample 5 is stored is sent thereto by mail, the test provider U3 sets the sample holder 6 in the test apparatus 1 and causes the test apparatus 1 to perform test processing. In another mode, when the plastic bag in which the sample 5 is stored is sent thereto by mail, the test provider U3 retrieves the gauze from the plastic bag and stores the gauze in the sample holder 6.

The test provider U3 can operate the management terminal 20 of the system of the test office 200 to access the Web page 31 of the broker 300. The test provider U3 can perform reception of a test request, acquisition of the information input to the questionnaire 7, uploading of the report 8 of the test result, and the like on the Web page 31 from the management terminal 20.

The test apparatus 1 has, as the mass spectrometer, a basic function of performing testing/body fluid analysis of the sample 5, and particularly has the function 1200 of determining the inappropriate sample in the first embodiment. The function 1200 determines/detects/outputs, during the test, whether or not the sample 5 to be tested is an inappropriate sample or a sample collected from a person other than the person of concern. The test apparatus 1 outputs the test result to the management terminal 20 or the server 30. The management terminal 20 or the server 30 produces the report 8 for notification of the test result from the test result. The report 8 is sent to the customer office 100.

The broker 300 acts as an intermediary between the customer office 100 where the test is received and the test office 200 that provides the test. The broker 300 coordinates with the test service provider to provide the simple drug test. A system of the broker 300 includes the server 30 and the like in the communication network 900, and uses the server 30 and the like to provide service as an input/output interface, a graphical user interface (GUI), and a contact point each related to the simple drug test service. The service includes the reception of the test request, inputting information to a questionnaire, notification of the test result, and the like. The broker 300 provides the Web page 31 as a GUI 31 using the server 30 or the like. In the embodiment, the GUI 31 (Web page 31) using the server 30 and the like is allowed to perform the reception of the test request, the inputting of information to the questionnaire, the notification of the test result, and the like.

Note that the broker 300 and a test service provider (test office 100) may also be integrated. In addition, the management terminal 20 and the test apparatus 1, which are separately provided in the test office 200, may also be integrated. While the management terminal 20 performs data management of information for the test provider U3 to perform the testing work, such data management may also be integrated with the PC 403 of the test apparatus 1.

The server 30 and the test apparatus 1 (particularly the PC 403 in FIG. 4) are connected appropriate by communication. Alternatively, it may also be possible that the test apparatus 1 and the management terminal 20 of the test provider U3 are connected by communication, while the management terminal 20 and the server 30 are connected by communication.

The questionnaire 7 basically includes filling in of a questionnaire form, mailing thereof, and the like, but is not limited thereto and, in the first embodiment, questionnaire data can be input on a screen of the Web page 31 of the server 30. Meanwhile, the report 8 basically includes filling in of a report form, mailing thereof, and the like, but is not limited thereto, and a report can be output on the screen of the Web page 31 of the server 30. It is not limited to the mode of the Web page 31, and e-mail or other modes can also be used. When a customer uses the Web page 31, mailing of the form or the like is unnecessary.

When the questionnaire 7 is to be in the mode of paper, a questionnaire form is mailed from the test office 200 or the broker 300 to the customer office 100, and the customer (e.g., the worker U2) inputs information to the questionnaire form and mails the questionnaire form to the test office 200 or to the broker 300. The test office 200 or the broker 300 acquires the information input to the questionnaire form therefrom. The system of the test office 200 or the broker 300 may also input the information input to the questionnaire form to a test subject information screen described later to digitize the information. Thus, questionnaire information, i.e., test subject information is obtained. The questionnaire information, i.e., the test subject information is transmitted to the management terminal 20 of the test office 200 or to the PC 403 of the test apparatus 1.

In a case of acquiring the questionnaire 7 in the mode of the Web page 31, the worker U2 at the customer office 100 accesses the Web page 31 from the work terminal 10 to input the questionnaire information on the screen of the Web page 31. The server 30 transmits the questionnaire information to the management terminal 20 of the test office 200 or to the PC 403 of the test apparatus 1 by communication. In other words, the management terminal 20 or the PC 403 references/acquires the questionnaire information from the server 30. The management terminal 20 may also transfer the questionnaire The function 1200 of the PC 403 of the test apparatus 1 uses the questionnaire information and a measurement result (mass measurement data) obtained by the test apparatus 1 from the sample 5 to determine whether the sample 5 is inappropriate and stores/outputs determination result information. The PC 403 may also transmit the determination result information to the management terminal 20 or to the server 30. The management terminal 20 may also transmit the determination result information to the server 30. The server 30 may also produce the report 8 on the basis of the determination result information.

Preferably, information on the sample 5 is written on the sample holder 6. The information to be written includes, e.g., an individual ID representing the test subject U1.

### Sequence of Simple Drug Test Service

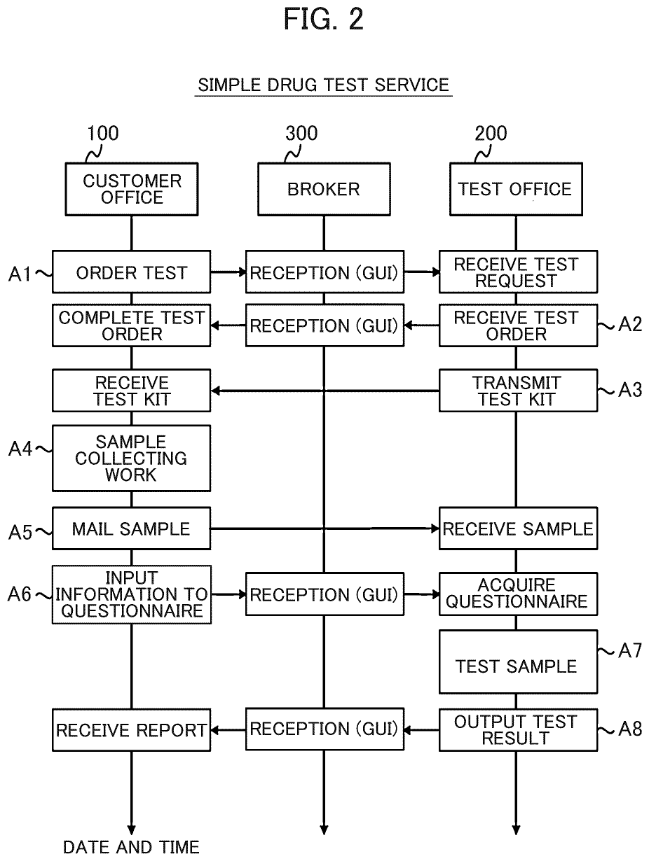

FIG. 2 illustrates a sequence of the simple drug test service between the components in FIG. 1. In Step A1, e.g., the worker U2 of the customer office 100 makes a request, i.e., places an order for the simple drag test with the test service provider 200 from the work terminal 10 via the Web page 31 of the GUI 31 of the broker 300 as the contact point. The test provider U3 of the test office 200 receives the request via the Web page 31.

In Step A2, the test provider U3 of the test office 200 receives the request for the test from the customer office 100 through the Web page 31. The worker U2 of the customer office 100 confirms/completes the test order through the Web page 31.

In Step A3, the test office 200 mails a test kit for collecting the sample 5 to the customer office 100 through the broker 300. The customer office 100 receives the test kit. The test kit is a set of tools for collecting the sample 5, and includes the sample holder 6 or a plastic bag. Together with the test kit, a questionnaire form may also be mailed.

In step A4, the worker U2 at the customer office 100 uses the test kit to perform work of collecting the sample 5 from the test subject U1 and storing the sample 5 in the sample holder 6 or the plastic bag. The worker U2 similarly collects the sample 5 from each of the one or more test subjects U1.

In Step A5, the worker U2 submits, e.g., the sample holder 6 after the collection by, e.g., mail to the test office 200 via the broker 300. The test office 200 receives the sample 5.

In Step A6, the worker U2 at the customer office 100 fills in the questionnaire form with the questionnaire information. Alternatively, the worker U2 accesses the Web page 31 on the work terminal 10 to input the questionnaire information to the screen of the Web page 31. In a case of the questionnaire form, the questionnaire form is mailed to the test office 100 via the broker 300.

In Step A7, at the test office 200, the test provider U3 uses the test apparatus 1 to perform work of testing the sample 5. The test provider U3 sets the sample holder 6 in the test apparatus 1, which introduces the sample holder 6 into the inside thereof and performs the test. The PC 403 of the test apparatus 1 obtains a test result. In the test processing in the test apparatus 1, the determination is also made by the function 1200 of determining the inappropriate sample.

In Step A8, the PC 403 of the test apparatus 1 transmits test result data to the management terminal 20 or to the server 30 of the broker 300. The test result data also includes a result of the determination by the function 1200 of determining the inappropriate sample. The management terminal 20 may also transmit the test result data to the server 30. The server 30 produces the report 8 for the notification of the test result from the test result data. The PC 403 or the management terminal 20 may also produce the report 8 from the test result data. The server 30 transmits the report 8 to the customer office 100. In a case where the report 8 is the report form, the report 8 is mailed. In a case where the report 8 is report data, the report data may be viewed on the Web page 31, or may also be transmitted by e-mail. The worker U2 at the customer office 100 can receive the report 8 and confirm the test result or the like.

### Outline of Simple Drug Test Service

FIG. 3 illustrates an outline of the simple drag test service, and particularly illustrates branches according to the result. In Step S1, at the customer office 100 at the location 1, the sample 5 is collected from the test subject U1. The worker U2 wipes off the body fluid containing sebum from a neck of the test subject U1 with gauze (i.e., wiper) to collect the body fluid as the sample 5. For example, the gauze is contained in the sample holder 6 or the plastic bag.

Note that the worker wears gloves or the like to perform work such as the wiping.

In Step S2, the sample holder 6 in which the sample 5 is stored is transported from the location 1 to the test office 100 at the location 2 by mail or the like. Alternatively, in Step S2, the questionnaire 7 is filled in with information on the test subject U1 and, when the questionnaire 7 is a form, the questionnaire 7 is mailed together with the sample 5 to the test office 100. Alternatively, the information input to the questionnaire 7, i.e., the test subject information is obtained via the Web page 31.

In Step S3, at the test office 200 at the location 2, the sample 5 (sample holder 6) from the customer office 100 and the questionnaire input information are acquired.

In Step S4, at the test office 200, on the basis of the work by the test provider U3, in the mass spectrometer which is the test apparatus 1, the sample holder 6 in which the sample 5 is stored is introduced, and the sample 5 is tested/analyzed. In the first embodiment, the test includes the determination of the inappropriate sample as processing by the characteristic function 1200. In a specific example, the PC 403 described later performs the processing by the Step S5 is outputting of the test result from the test apparatus 1, which is branched according to the rest result. Step S6 is performed in a case where the sample is roughly determined to be appropriate as one determination result. Step S7 is performed in a case where the sample is roughly determined to be inappropriate as another determination result, i.e., a case where it is determined that the sample 5 has not been collected from the person of concern, and there is a possibility of switching, mix-up, or the like of the sample.

In the case of Step S6, the test results include a case where the test result in Step S8 is a pass and an illegal drug or the like was not detected as a component substance of the sample 5 and a case where the test result in Step S9 is a fail and an illegal drug or the like is detected as the component substance of the sample 5. In the case where the test result in Step S9 is a fail, a detailed examination is consequently requested in Step S10.

Meanwhile, in the case where the sample is determined to be inappropriate in Step S7, sample confirmation and a re-test are requested as a coping method in Step S11. The sample confirmation is a confirmation by the test provider U3, which is a reconfirmation of the sample 5 set in the test apparatus 1. A request for the re-test includes a request/recommendation to the customer office 100, which is a request for re-testing of the sample 5 newly collected from In a case of any branch (S8, S10, or S11), then, in Step S12, a notification of the result is eventually given to the customer office 100 according to each case. The test office 200 produces the report 8 for the notification of the test result including the determination result, and transmits the report 8 to the customer office 100. The report 8 may be mailing of the report form, or may also be provision of report data on the Web page 31 of the broker 300. The customer office 100 side receives the report 8 for the notification of the result. This completes a series of sequences.

Note that, in a case of the detailed examination in Step S10, the detailed examination may also be performed at another test office or specialized institution (e.g., hospital) other than the test office 200. In that case, when the detailed examination is requested, the other test office or specialized institution is introduced.

In a single test, a predetermined number of target component substances can be analyzed. In this test, even when the test subject U1 has been using an illegal drug for, e.g., the past one month, the illegal drug can be detected.

On the Web page 31 as the GUI 31, a series of schedules in the simple drug test as in FIG. 2 and FIG. 3 described above or the like can also be checked. Examples of the schedules include by when the sample should be mailed, when the report 8 of the result will be available, and the like.

Note that FIG. 1 illustrates the only one test subject U1 but, in a case where there are a plurality of the test subjects U1 at the customer office 100, it is possible to collectively request testing of the samples 5 from the plurality of people and collectively obtain the test results from the plurality of people as the report 8.

### Outer Appearance of Test Apparatus

FIG. 4 illustrates an example of a configuration of an outer appearance of the test apparatus 1. The test apparatus 1 includes a main body 401, a cover 402, the PC 403, and the like. FIG. 4 (A) illustrates a state where the cover 402 is closed during normal use, which is a state where the PC (computer) 403 is placed on the cover 402 used as a base. FIG. 4 (B) illustrates a state where the cover 402 is opened during maintenance work or the like, which is a state where a component 404 (described later) placed inside the main body 401 is visible. The PC 403 is the computer serving as a controller for operation of the test apparatus 1, an input thereto, and an output therefrom, which has a data processing function and provides a GUI when the test provider U3 (FIG. 1) or the like operates the test apparatus 1.

By operating the PC 403, the test provider U3 can switch the operation of the test apparatus 1 between ON/OFF states and conduct testing, setting, maintenance, and the like. To the PC 403, a typical notebook PC or the like can be applied, but the PC 403 is not limited thereto. The PC 403 includes a processor, a memory, a communication interface, an input device, an output device, and the like, and processes a detection signal obtained from the main body 401 or the like to obtain mass measurement data and test result data. The PC 403 holds data such as the mass measurement data in the memory, and transmits the data to the outside at an appropriate time.

The PC 403 of the test apparatus 1 references the questionnaire input information, i.e., the test subject information as information related to the testing of the sample 10 from the server 30 or the management terminal 20. The PC 403 integrates the test result from the test apparatus 1 and the determination result from the function 1200. Test result data also includes determination result data. The PC 403 transmits the test result data to the management terminal 20 or to the server 30 at an appropriate time.

When the sample is determined to be inappropriate as a result of the determination by the function 1200 of determining the inappropriate sample, the PC 403 of the test apparatus 1 may also perform control such that the test processing in the main body is temporarily suspended.

### Functional Block as Test Apparatus

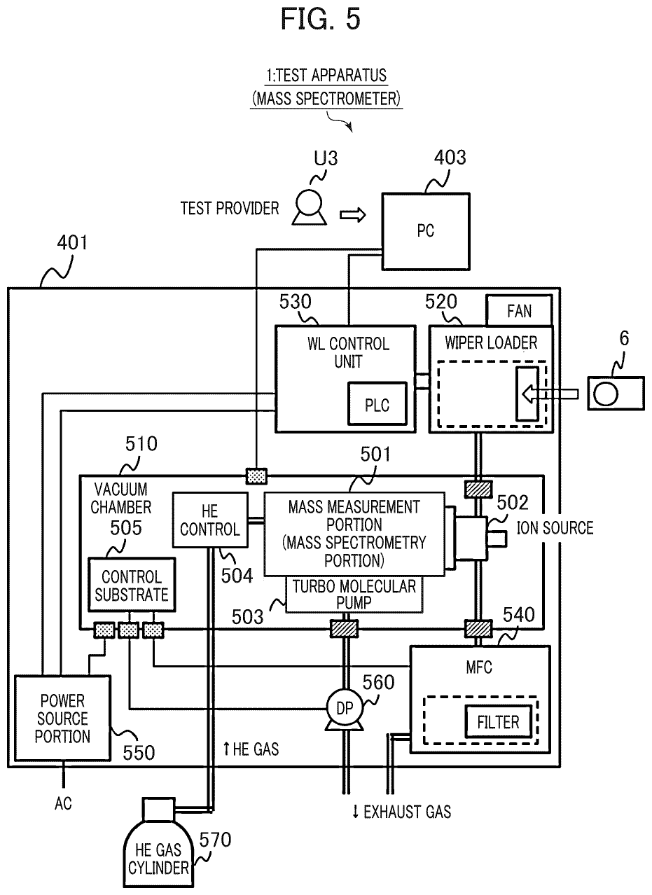

FIG. 5 is a diagram illustrating a functional block configuration of the test apparatus 1. In FIG. 5, the cover 402 is omitted. FIG. 5 also illustrates the test provider U3. For example, in a case of the simple drug test by the off-site monitoring, the test provider U3 sets the sample holder 6 (i.e., wiper holder) in which the sample 5 is stored in a wiper loader 520. FIG. 5 illustrates a case where the test apparatus 1 is the mass spectrometer including an ion-trapping mass measurement portion 501 with an automatic positive/negative polarity switching function.

The test apparatus 1 includes, in the main body 401, a vacuum chamber 510, the wiper loader 520, a wiper loader (WL) control unit 530, a mass flow controller (MFC) 540, a power source portion 550, a discharge pump (DP) 560, a He gas cylinder 570, and the like. The vacuum chamber 510 includes the mass measurement portion (i.e., mass analysis portion) 501, an ion source 502, a turbo molecular pump 503, a He control portion 504, a control substrate 505, and the like.

The wiper loader 520 is a sample introduction portion into which the sample holder 6 in which the wipe-off sample 5 is stored is to be introduced and which has an input port opening in the front of the apparatus and a fan, and the sample holder 6 holding the sample 5 is introduced into the wiper loader 520. From the introduced sample holder 6, a sample gas corresponding to the sample 5 is introduced into the vacuum chamber 510.

The wiper loader (WL) control unit 530 is a unit which includes a PLC (programmable logic controller) and the like, and controls an operation of the wiper loader 520.

The mass flow controller (MFC) 540 is a unit which controls a flow of a gas such as the sample gas or a buffer gas (e.g., He gas). The MFC 540 is connected to the control substrate 505 to control an air pump, a flow rate, a pressure, and the like on the basis of control by the control substrate 505. The MFC 540 has a filter opening in the front of the apparatus and the like to exhaust the extra sample gas to the outside.

The turbo molecular pump 503 is connected to the mass measurement portion 501 to perform evacuation. The discharge pump (DP) 560 is connected to the turbo molecular pump 503 to perform evacuation to the outside on the basis of the control by the control substrate 505.

The He gas cylinder 570 supplies a He gas to the He control portion 504 via piping. The He gas cylinder 570 is illustrated as a component of the test apparatus 1, but may also be considered to be an object placed at the outside of the test apparatus 1. The He control portion 504 supplies the He gas supplied from the He gas cylinder 570 to the mass measurement portion 501.

The power source portion 550 generates DC power on the basis of an AC power source input from the outside and supplies the required power to each of the portions.

In the vacuum chamber 510, the control substrate 505 controls the components of the vacuum chamber 510 and the like. The control substrate 505 corresponds to a controller that performs main control of measurement in the test apparatus 1. The control substrate 505 receives an instruction from the PC 403. The PC 403 can give an instruction to the WL control unit 530, the control substrate 505 of the vacuum chamber 510, or the like.

The mass measurement portion (mass analysis portion) 501 measures a mass spectrum for the ionized sample supplied through the ion source 502, details of which will be described later. A signal for the mass spectrum measured by the mass measurement portion 501 is sent to the PC 403 to be subjected to data processing.

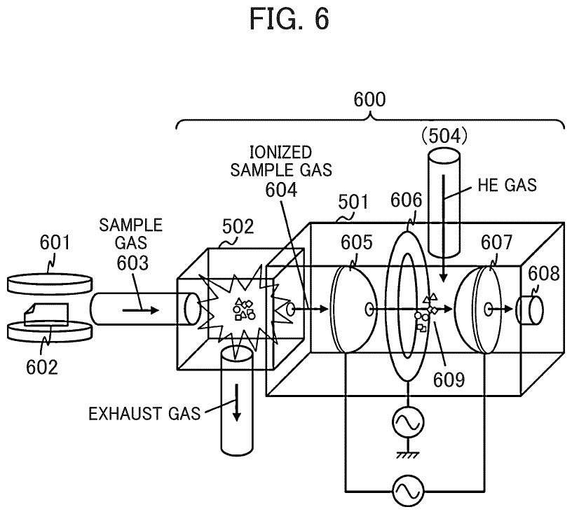

An outline of the mass measurement in the test apparatus 1 is as follows. The test provider U3 introduces the sample holder 6 into the wiper loader 520. The wiper loader 320 supplies, on the basis of the control by the WL control unit 530, the sample gas corresponding to the sample 5 to the ion source 502 of the vacuum chamber 510. Specifically, the wiper loader 520 generates a sample gas 603 from the sample 5 on a gauze (wiper) 602 of a sample introducer 601 (FIG. 6), and the sample gas 603 is supplied, on the basis of the control by the MFC 540, to the ion source 502 of the vacuum chamber 510 through the piping.

In the ion source 502, the sample gas 603 is ionized. An ionized sample gas 604 is supplied into a chamber of the mass measurement portion 501. The extra sample gas is exhausted to the outside via the MFC 540. In the mass measurement portion 501, the introduced ionized sampled gas 604 is subjected to mass separation and structural analysis. Details thereof are illustrated in FIG. 6.

As a result of measurement by the mass measurement portion 501, a mass spectrum measurement signal is obtained. The measurement signal (i.e., detection signal) is sent to the PC 403 and subjected to data processing, with the result that mass spectrum measurement data (mass measurement data) is obtained. In the PC 403, on the basis of the mass measurement data and inner database information, the target component substance of the sample 5 is identified, and detection result data is generated.

### Mass Measurement Portion of Test Apparatus

FIG. 6 illustrates an example of a configuration of the mass measurement portion 501 or the like in FIG. 5. The mass measurement portion 501 and the ion source 502 are included in an ion optical system 600. The mass measurement portion 501 has, in the chamber thereof, end cap electrodes 605 and 607 (i.e., lens electrodes) and a ring electrode 606. The ionized sample gas 604 (i.e., ions) introduced in the chamber is detected as a detection signal from a detector 608 sequentially through the end cap electrode 605, the ring electrode 606, and the end cap electrode 607. To the end cap electrodes 605 and 607, a high-frequency voltage is applied. To the ring electrode 502, the high-frequency voltage is applied. In the present configuration example, the ion trapping type having the ring electrode 606 is shown, but a linear ion trapping type having a linear electrode may also be used.

In the present configuration example, ions 609 are held by a trap configured to include the end cap electrodes 605 and 607 and the ring electrode 606. When the high-frequency voltage at the ring electrode 606 is increased, the ions 609 having smaller m/z are emitted earlier to be detected through the detector 608. Thus, as a result of the separation of the ions 609 corresponding to the component according to m/z, a mass spectrum is obtained.

### Mass Spectrum

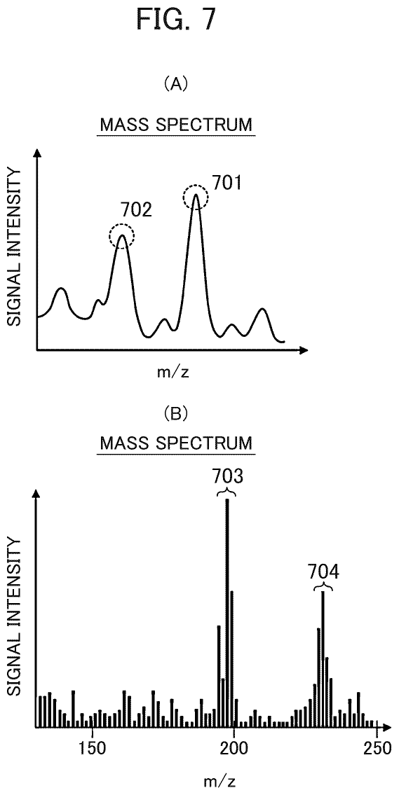

FIG. 7 illustrate an example of a mass spectrum obtained as a result of measurement/analysis by the mass measurement portion 501 of the test apparatus 1. FIG. 7 (A) illustrates an example of the mass spectrum obtained as a result of the measurement in a waveform. An abscissa axis represents m/z, while an ordinate axis represents a signal intensity. Since a sample gas contains a plurality of component substances, in the mass spectrum, a plurality of peaks (e.g., peaks 701 and 702) exist in mixed relation. For example, by comparing a signal intensity integrated value within a measurement range around a peak value of m/z to a threshold, it is possible to determine whether or not each of the component substances corresponds to a predetermined target component substance.

FIG. 7(B) illustrates another example of the mass spectrum. In this example, e.g., a peak 703 and a peak 704 can be monitored. The peak 703 is in the vicinity of m/z 200, while the peak 704 is in the vicinity of m/z 230. An example of the component substance corresponding to the peak 703 is trichlorophenol. An example of the component substance corresponding to the peak 704 is tetrachlorophenol.

### Computer System: PC

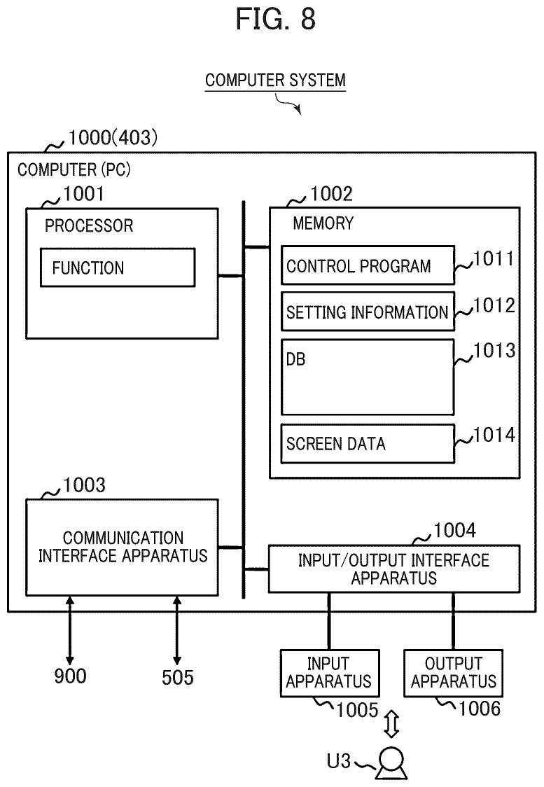

FIG. 8 illustrates an example of a configuration of a computer system related to the PC 403 of the test apparatus 1. The computer system in FIG. 8 has a computer 1000, while having an input apparatus 1005 and an output apparatus 1006 each connected to the outside of the computer 1000. The predetermined user U3, who is the test provider U3, operates the input apparatus 1005 and the output apparatus 1006 or the management terminal 20 as a client terminal to thereby use functions of the PC 403 as the computer system.

The computer 1000 includes a processor 1001, a memory 1002, a communication interface apparatus 1003, an input/output interface apparatus 1004, and the like, which are connected to each other via an architecture such as a bus. The processor 1001 is configured to include the CPU, a ROM, a RAM, and the like. The memory 1002 is configured to include a nonvolatile storage apparatus, an auxiliary storage apparatus, and the like. The communication interface apparatus 1003 includes a communication interface with the communication network 900 and a communication interface corresponding to the control substrate 505 of the main body 401 of the test apparatus 1 or the like. To the input/output interface apparatus 1004, the input apparatus 1005 and the output apparatus 1006 are connected. Examples of the input apparatus 1005 include a keyboard and a mouse, while examples of the output apparatus 1006 include a display and a printer. The input apparatus 1005 and the output apparatus 1006 may also be configured to include a touch panel.

In the memory 1002, for example, a control program 1011, setting information 1012, a database (DB) 1013, screen data 1014, and the like are stored. The control program 1011 is a program which implements a predetermined function. The processor 1001 executes processing according to the control program 1011 read from the memory 1002 to thereby implement the predetermined function as an execution module. The setting information 1012 is system setting information and user setting information each related to the control program 1011. The DB 1013 is a database in which, e.g., data for analyzing the target component substance, the test result data, and the like is stored. The screen data 1014 is data to be displayed on a screen of the display apparatus serving as the output apparatus 1006.

Note that, by using the PC 403 as a server, the management terminal 20 in FIG. 1 or the like may also be connected as a client terminal via a LAN to form a client server system. Each of the apparatuses in FIG. 1 may also be similarly configured in a computer system as illustrated in FIG. 8.

### Computer System: Server

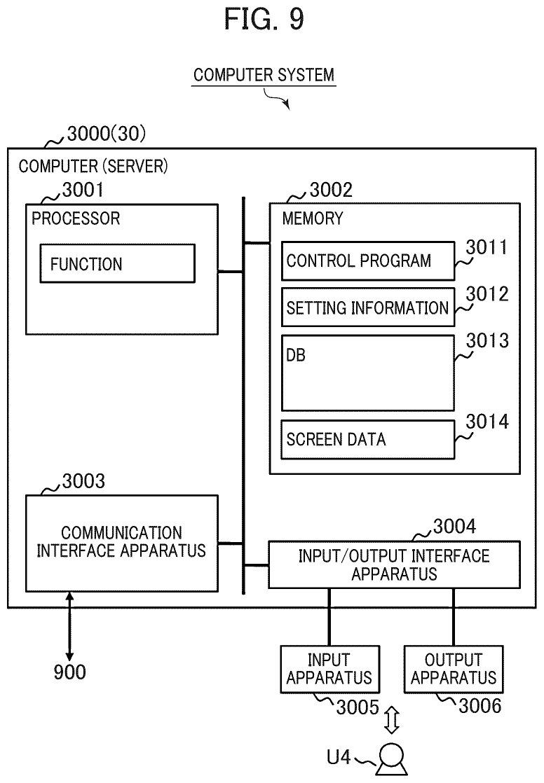

FIG. 9 illustrates an example of a configuration of a computer system related to the server 30. The computer system in FIG. 9 has a computer 3000, while having an input apparatus 3005 and an output apparatus 3006 each connected to the outside of the computer 3000. A predetermined user U4, who is the administrator U4 of the broker 300, operates the input apparatus 3005 and the output apparatus 3006 or the client terminal to thereby use functions of the server 30 as the computer system.

The computer 3000 includes a processor 3001, a memory 3002, a communication interface apparatus 3003, an input/output interface apparatus 3004, and the like, which are connected to each other via an architecture such as a bus. The processor 3001 is configured to include the CPU, the ROM, the RAM, and the like. The memory 3002 is configured to include a nonvolatile storage apparatus, an auxiliary storage apparatus, and the like. The communication interface apparatus 3003 includes the communication interface with the communication network 900. To the input/output interface apparatus 3004, the input apparatus 3005 and the output apparatus 3006 are connected.

In the memory 3002, for example, a control program 3011, setting information 3012, a database (DB) 3013, screen data 3014, and the like are stored. The control program 3011 is a program which implements a predetermined function. The processor 3001 executes processing according to the control program 3011 read from the memory 3002 to thereby implement the predetermined function as an execution module. The setting information 3012 is system setting information and user setting information each related to the control program 3011. The DB 3013 is a database in which data for, e.g., the questionnaire 7, the report 8, and the like is to be stored.

The screen data 3014 is data for displaying the Web page 31.

FIG. 10 illustrates a flow of the test (i.e., measurement/analysis or the like) in the test apparatus 1. In Step S101, in the test apparatus 1, the testing of the sample 5 is started. For example, the test provider U3 checks information on the sample 5 from the test subject on the screen of the PC 403, sets the sample holder 6 in the test apparatus 1, and then presses a measurement start button on the screen of the PC 403.

In Step S102, the test apparatus 1 controls a mechanism as described previously (FIG. 5 and FIG. 6) to measure the mass spectrum of the sample 5 and thereby obtain mass measurement data. In other words, as a component amount related to the target component substance, data of a signal intensity versus m/z as in the example of FIG. 7 is obtained. The PC 403 obtains the mass measurement data and stores the obtained data in the memory.

In Step S103, the PC 403 performs processing due to the function 1200 of determining the inappropriate sample. Details thereof will be described later. At the time of the determination, the PC 403 also references the questionnaire input information (i.e., the test subject information) required for the determination as well as information on the respective reference ranges for the individual determination conditions. When each of the component amounts in the measurement result is compared to the reference range for the determination condition and the component amount is within the reference range, the determination in Step S103 substantially corresponds to clearing the determination condition, as will be described later.

Step S104 is a branch according to a result of the determination by the function 1200. When the sample is determined to be inappropriate (fail), the processing advances to Step S105 where the sample is confirmed and the re-test is requested. When the sample is determined to be appropriate (pass), i.e., when the sample does not meet the condition for determining the inappropriate sample, the processing advances to Step S106.

In Step S106, a test result of the simple drug test is determined. For example, when a component amount of an illegal drug exceeding a threshold is detected as the target component substance, the test result is a fail, i.e., detection of the drug. Step S7 is a branch according to the test result. When there is the detected drug, the processing advances to Step S108, and a detailed examination is requested. When there is no drug detected, the processing advances to Step S109.

In Step S109, the testing of the sample 5 in the test apparatus 1 is ended. The test apparatus 1 outputs a result of testing the sample 5 (including the result of the determination by the function 1200). Note that, when there is another sample 5 as one of a plurality of the samples 5 at the customer office 100, the test is similarly repeated. In Step S110, the PC 403 of the test apparatus 1, the management terminal 20, or the server 30 produces the report 8 on the basis of test result data. In the present example, the server 30 produces the report 8 based on the test result. The broker 300 sends a report form corresponding to the report 8 to the customer office 100 by mail. Alternatively, the broker 300 posts the Web page 31 corresponding to the report 8, and the customer office 100 views the report 8 on the Web page 31.

### Questionnaire

Acquisition of information of the questionnaire 7 may be in a mode in which a questionnaire form is provided together with a test kit such that the questionnaire form is filled in and returned, or may also be in a mode in which the questionnaire is filled in on the Web page 31. In the former case, it may be possible to read the information with which the questionnaire form is filled in by OCR or the like and acquire the information as the questionnaire input information, or it may also be possible for a person at the broker 300 to input the information with which the questionnaire form is filled in on a screen described later. In either of the modes, the acquired questionnaire input information, i.e., the test subject information is input to the PC 403 of the test apparatus 1.

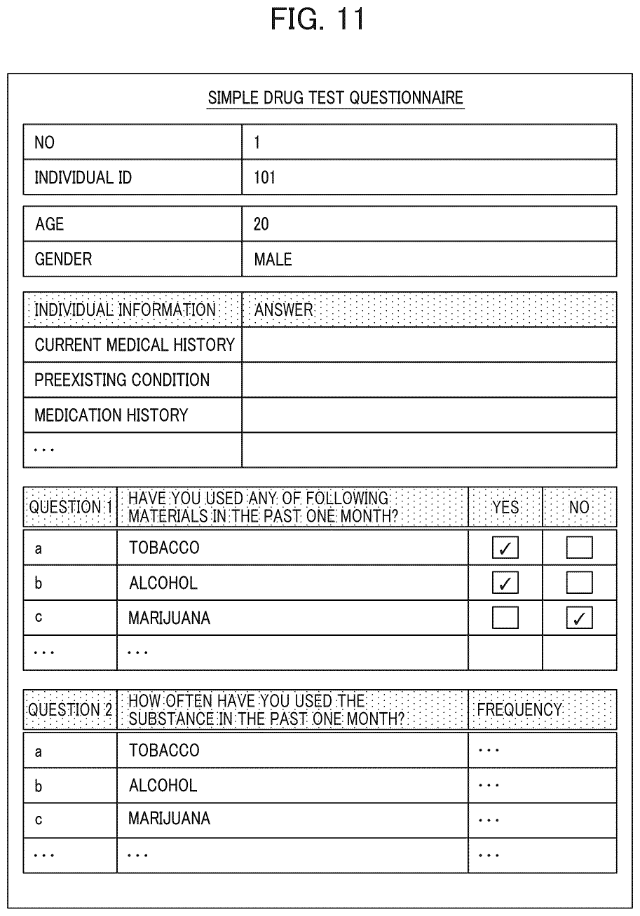

FIG. 11 illustrates, as an example of a configuration of the questionnaire 7, an example of a configuration of the questionnaire form. The questionnaire form in FIG. 11 has entry fields for an individual ID, an age, and a gender corresponding to the test subject U1. In addition, the questionnaire form in FIG. 11 has entry fields for a current medical history, a preexisting condition, and a medication history. The questionnaire form in FIG. 11 also has, as a question 1, a question such that “HAVE YOU USED ANY OF FOLLOWING DRUGS IN THE PAST ONE MONTH?”, and also has an entry field for each of drugs such as tobacco, alcohol, marijuana, and stimulant, in which an answer to a question about drug use is to be written with YES or NO. In addition, the questionnaire form in FIG. 11 has a field in which, when any of the drugs in the question 1 has been used, how often the drug has been used is to be written as an answer to a question 1.

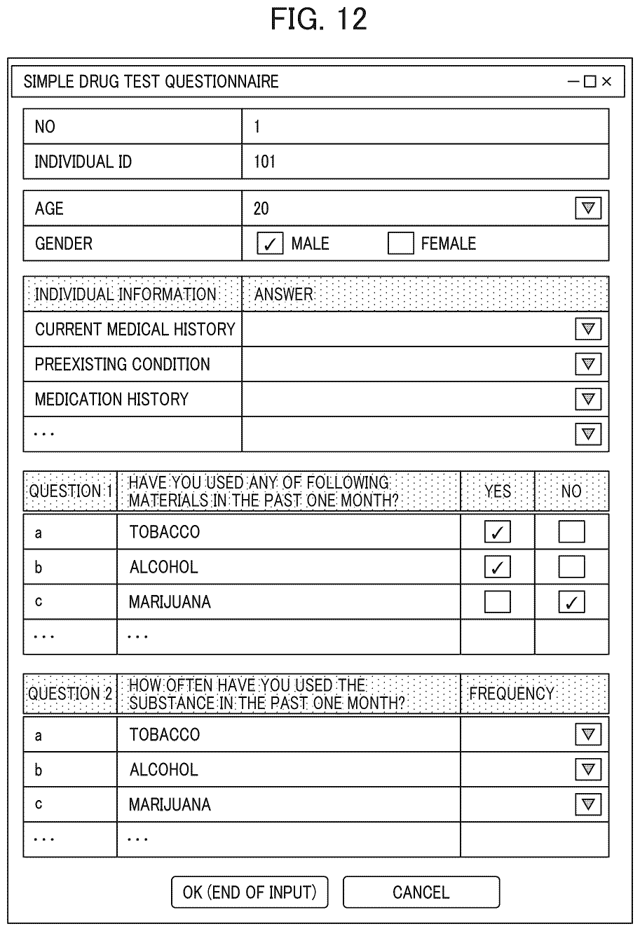

FIG. 12 illustrates an example of a configuration of a questionnaire input screen on the Web page 31 as an example of a configuration of the questionnaire 7. The screen in FIG. 12 is provided with the same items as those in FIG. 11, and information can be input by using a GUI such as a check box, a list box, or a text box.

### Test Subject Information Input Screen

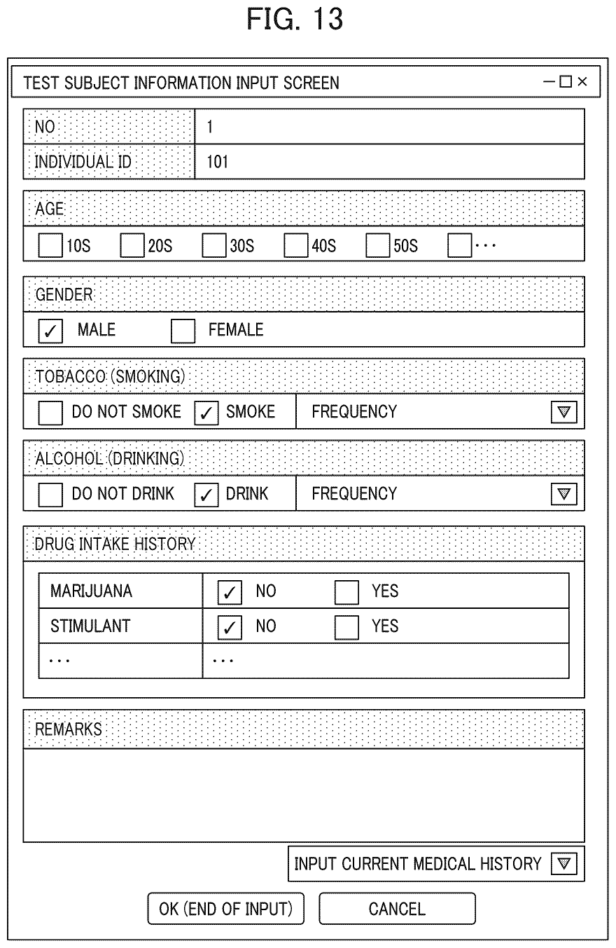

FIG. 13 illustrates an example of a configuration of the test subject information input screen in the system of the broker 300 when the test subject information is to be input on the basis of the information in the questionnaire form. The broker 300 inputs the test subject information to a screen as in FIG. 13 on the basis of the information in the questionnaire form acquired from the customer office 100. Note that, when the questionnaire information has been input in a Web format as in FIG. 12 from the beginning, it may also be possible to omit inputting on the present screen, and directly use the questionnaire information.

To the test subject information input screen in FIG. 13, e.g., the administrator U4 at the broker 300 inputs the test subject information on the basis of the questionnaire information. The screen in FIG. 13 has the same items as those of the questionnaire information in each of FIG. 11 and FIG. 12. A basic input method allows required information to be easily input by checking/unchecking of a check box for each of the items on the screen by clicking or the like, selection of any among options defined in a list box, or the like.

When there are a current medical history, a medication history, a drug intake history, and the like of the test subject U1, the information can also be written in a remark column. In case of a detailed examination due to detection of an illegal drug or the like, the test subject information on the present screen can be provided/used.

The function 1200 of determining the inappropriate sample handles the target component substances from individual viewpoints under a plurality of determination conditions described later. As a result, attribute information of the test subject U1 corresponding to the sample 5 or the like is necessary. Accordingly, the present test system acquires, prior to the test, questionnaire input information as the attribute information of the test subject U1 together with the sample 5. In the questionnaire input information, information such as, e.g., the age, the gender, the smoking status, and the drinking status is acquired. The present test system uses the acquired questionnaire information, i.e., the test subject information for the determination by the function 1200. The function 1200 makes the determination by using the questionnaire information together with the mass measurement data as a measurement result in the test.

The questionnaire information obtained in the present test system can also be provided/used at the time of the detailed examination.

Note that, in a case were the questionnaire 7 has no input information, even when the information on the test subject U1 is not input on the test screen, the test can be provided by the test apparatus 1. In that case, processing related to the item having no input is skipped.

### Report

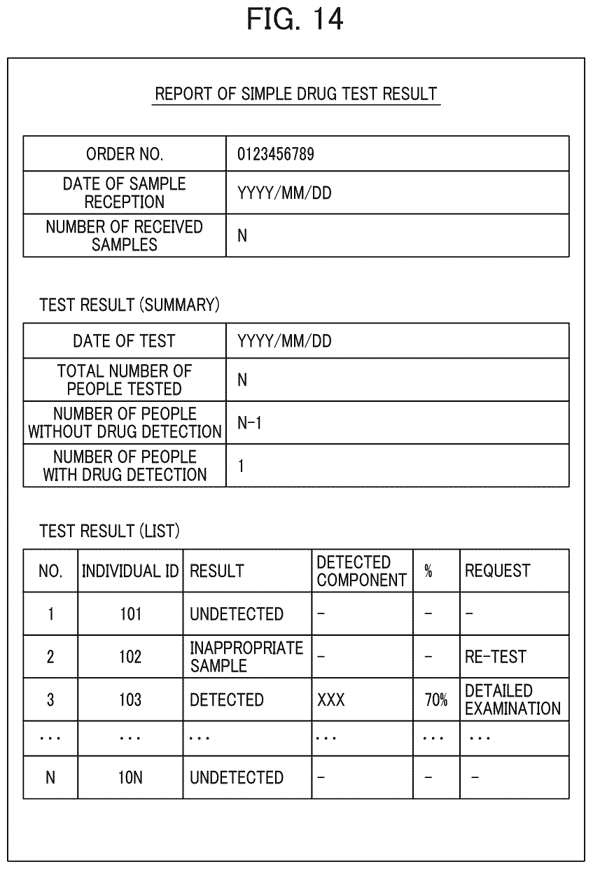

FIG. 14 illustrates, as an example of the report 8 for the notification of the test result, an example of a configuration in a case of a report form. The report form in FIG. 14 lists information such as an order form number, a date of sample reception, and the number of received samples. The report form in FIG. 14 also lists, as a “TEST RESULT (SUMMARY) ”, information such as a date of test, the total number of people tested, the number of people without drug detection, and the number of people with drug detection. In addition, the report form in FIG. 14 lists, as a “TEST RESULT (LIST)”, information such as a result, a detected component, %, and a request for each individual ID of the test subject U1. In the result item, as a result of testing an illegal drug or the like, “DETECTED (FAIL)” or “UNDETECTED (PASS)” is listed. In addition, in the result item, when the inappropriate sample is determined as a result of the determination by the function 1200 of determining the inappropriate sample, “INAPPROPRIATE SAMPLE” is listed. In the detected component item, a component substance detected when an illegal drug or the like is detected is listed. In the % item, information related to accuracy of detection when an illegal drug or the like is detected, e.g., information on a result of estimating, e.g., how long ago in the past the drug was ingested is listed. In the request item, as a request according to the result, a request for a detailed examination is listed in case of “DETECTION”, while a request for the re-test is listed in case of “INAPPROPRIATE SAMPLE”.

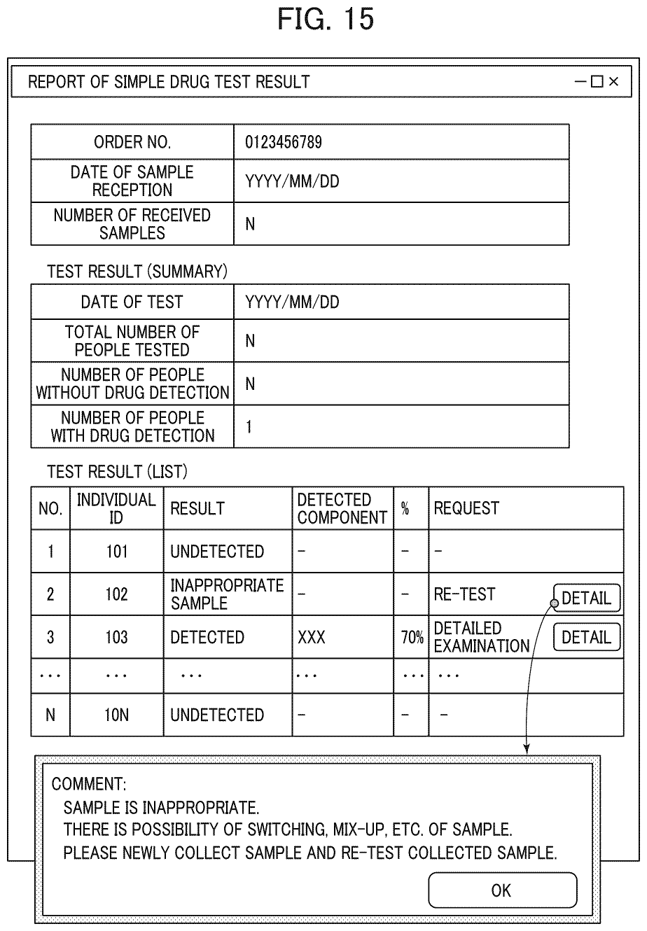

FIG. 15 illustrates, as an example of the report 8 for the notification of the test result, an example of a configuration in a case of a screen of report data on the Web page 31. The screen in FIG. 15 displays information on the same items as those in FIG. 14. In addition, on the screen in FIG. 15, the request item is provided with, e.g., a detail button and, when the user presses the detail button, detailed information is displayed and can be checked. For example, when the detail button is pressed in the row in which the result is “INAPPROPRIATE SAMPLE”, as illustrated in the drawing, a comment on the result of determining the “INAPPROPRIATE SAMPLE” and on the request for “RE-TEST” is displayed. For example, the comment is a message such that “SAMPLE IS INAPPROPRIATE. THERE IS POSSIBILITY OF SWITCHING, MIX-UP, ETC. OF SAMPLE. PLEASE NEWLY COLLECT SAMPLE AND RE-TEST COLLECTED SAMPLE”. It may also be possible to cause a transition from the detail button to a screen for requesting With regard to the function 1200, the report 8 for the notification of the test result is possible without being limited to the example of an alert output of “INAPPROPRIATE SAMPLE” in FIG. 14 and FIG. 15.

### Customer ID Management

The system of the broker 300 or the test office 200 performs management of customer IDs. This is to ensure that there is no switching of the sample 5 or the like on the service provider side. The system of the broker 300 or the test office 200, e.g., the server 30 performs data management of information including information (customer office ID) for identifying the customer office 100 and information (test subject ID and the individual ID described above) for identifying the test subject U1.

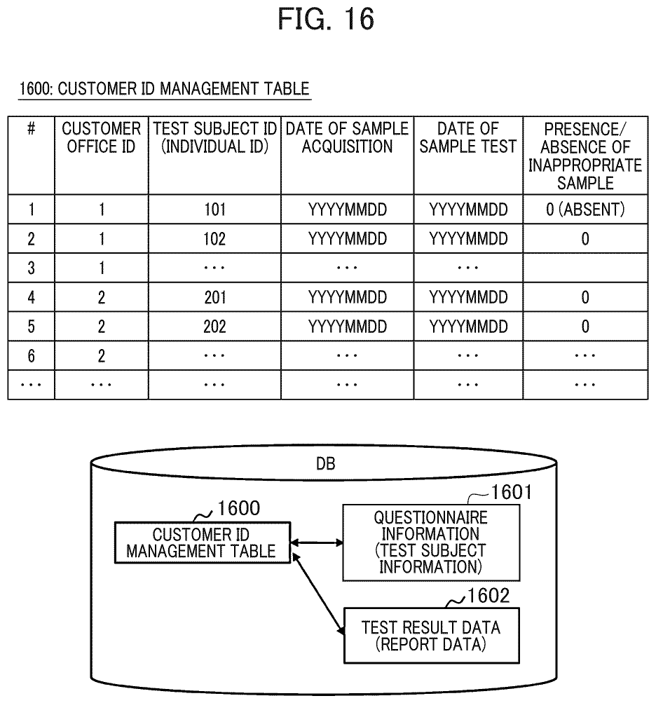

FIG. 16 illustrates an example of a customer ID management table on the service provider side. A customer ID management table 1600 in FIG. 16 has, as items, the customer office ID related to a plurality of the customer offices 100, a test subject ID related to a plurality of the test subjects U1, a date of sample acquisition, a date of sample test, and the presence or absence of an inappropriate sample. The date of sample detection is a date and a time when the test office acquired the sample 5. The date of sample test is a date and a time when the test office tested the sample 5. The presence or absence of an inappropriate sample is determined on the basis of whether or not the sample has been detected to be inappropriate with regard to the function 1200.

The service provider, e.g., the server 30 associates various data including the customer ID management table 1600 as described above, the questionnaire information (i.e., test subject information) 1601, and the test result data 1602 (or report data) with each other, and manages the data in a DB (database) or the like.

### Function of Determining Inappropriate Sample

FIG. 17 illustrates, as a characteristic function in the PC 403 as the controller of the test apparatus 1, an outline of the function 1200 of determining the inappropriate sample. The PC 403 implements a testing function 1100 and the function 1200 of determining the inappropriate sample through execution of processing according to a control program in the memory 1002 by the processor 1001 on the basis of a base configuration as in FIG. 8 described above.

The test apparatus 1 has the basic testing function 1100. The testing function 1100 is a function of determining that, when a component amount (signal intensity versus m/z in the mass spectrum described above) of the target component substance, which is a predetermined illegal drug or the like, is exceeding a predetermined value (i.e., a reference value, an upper limit value, or the like), there is a possibility of ingesting the illegal drug or the like, delivering a fail (with detection) as a test result, and requesting the detailed examination.

During test processing, the testing function 1100 obtains, as measurement result data, mass measurement data 1701 on the basis of a detection signal (i.e., measurement signal) from the control substrate 505 of the test apparatus 1. The testing function 1100 determines the test result on the basis of the mass measurement data 1701 and target component substance data 1702 set in advance, and produces test result data 1703.

During the test processing, the function 1200 of determining the inappropriate sample references the mass measurement data 1701, test subject information 1704 as the questionnaire information, and reference range information 1705 to determine whether or not the sample is inappropriate.

The function 1200 causes the determination result to be included in the test result data 1703 resulting from the testing function 1100.

The PC 403 stores the test result data 1703 including the result of the determination by the function 1200, and outputs the test result data 1703 to the management terminal 20 and to the server 30. Note that the test result data resulting from the testing function 1100 and the determination result data due to the function 1200 may be generated as separate data sets, or may also be generated as a single data set.

The function 1200 of determining the inappropriate sample is, i.e., a sample determining function, and is also a function of determining a possibility of switching, mix-up, or the like. This function 1200 includes at least four functions, i.e., determination conditions. The function 1200 has a function 1201 of determining whether or not the sample is consistent with an attribute such as an age or a gender as a determination condition 1, a function 1202 of determining whether or not the sample has been collected from a human being as a determination condition 2, a function 1203 of determining whether or not the sample is consistent with smoking effects as a determination condition 3, and a function 1204 of determining whether or not the sample is consistent with drinking effects as a determination condition 4.

In the first embodiment, the function 1200 makes a determination about each of the four determination conditions for the foregoing four functions, and determines the sample to be inappropriate when at least one of determination results meets the determination condition, i.e., when at least one of determination results falls outside a reference range described later. In other words, when none of the determination results meets the four determination conditions, i.e., when all the determination results fall within the reference ranges, the sample is determined to be appropriate. The inappropriate sample is, i.e., estimation of the sample not collected from the person of concern or a possibility of switching, mix-up, or the like.

### Function of Determining Inappropriate Sample: Determination Condition

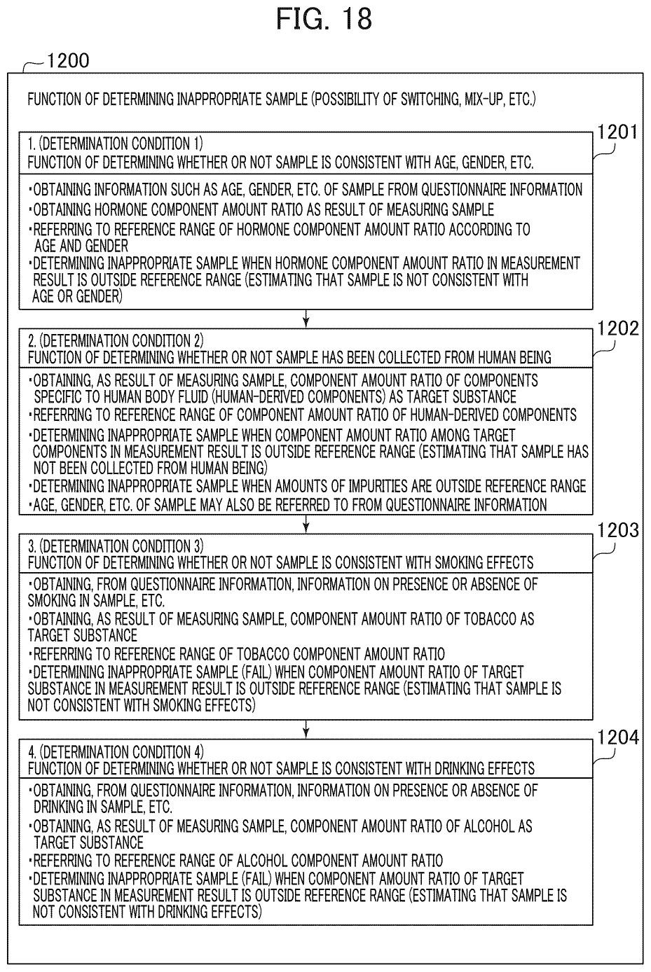

FIG. 18 illustrates, as details of the function 1200 of determining the inappropriate sample, details of the four functions 1201 to 1204 for the four determination conditions. The details of the individual functions are as follows.

Determination Condition 1: The function 1200 has a function 1201 of determining whether or not the sample is consistent with an attribute such as an age or a gender: This function 1201 references, from the questionnaire information 1704, the attribute information of the test subject U1 corresponding to the target sample 5, such as the age or gender. The function 1201 obtains, as a result of measuring the sample 5, a hormone component amount ratio from the mass measurement data 1701. The function 1201 references, from the reference range information 1705, a reference range of the hormone component amount ratio corresponding to the age and gender of the sample 5. This function 1201 compares the hormone component amount ratio in the measurement result to the reference range to be referenced, and determines the sample to be inappropriate when the hormone component amount ratio is outside the reference range. In other words, the result of the determination by this function 1201 corresponds to estimation that the measured sample 5 is not consistent with the age, gender, and the like of the test subject U1.

For example, males and females have different types of hormones and different hormone balances. In addition, for example, a male in his 10s and a male in his 50s also have different types of hormones and different hormone balances. When the sample 5 has been collected from a person having an attribute different from that of the person of concern, a difference appears in the component amounts of the target component substance. Accordingly, by examining the hormone component amount ratio, the function 1201 can estimate whether or not the sample 5 is consistent with the attribute of the test subject of concern and whether or not there is a possibility that the sample 5 has been collected from a person having an attribute different from that of the person of concern.

Determination Condition 2: The function 1202 of determining whether or not the sample has been collected from a human being: This function 1202 obtains, as a result of measuring the sample 5, the component amount ratio in a component substance (i.e., human-derived substance) specific to a human body fluid as the target component substance from the mass measurement data 1701. This function 1201 references a reference range of the component amount ratio in the human-derived target component substance from the reference range information 1705. This function 1202 compares the component amount ratio in the human-derived target component substance in the measurement result to the reference range to be referenced, and determines the sample to be inappropriate when the component amount ratio is outside the reference range. In other words, the result of the determination by the function 1202 corresponds to estimation that the measured sample 5 is not a sample collected from a human-being.

In a case of a sample collected from any object other than the test subject of concern who is a human being, e.g., when the sample has been wiped off from a wall, a desk, a bag, clothes, an animal, food and drink, or the like, component amounts of human-derived components in the sample are mostly zero or small amounts, and impurities other than the human-derived components are oftentimes contained in large amounts. Therefore, by examining the amounts of the human-derived components and the amounts of the impurities, the function 1202 can estimate whether or not the sample 5 has been collected from the test subject of concern.

Determination Condition 3: The function 1203 of determining whether or not the sample is consistent with smoking effects: This function 1203 references, from the questionnaire information 1704, information on a situation such as a smoking status of the test subject U1 corresponding to the target sample 5. This function 1203 obtains, as the result of measuring the sample 5, a component amount ratio in tobacco as the target component substance from the mass measurement data 1701. This function 1203 references, from the reference range information 1705, a reference range of the component amount ratio in the sample 5, which is tobacco, as the target component substance. This function 1203 compares the component amount ratio in tobacco as the target component substance in the measurement result to the reference range to be referenced, and determines the sample to be inappropriate when the component amount ratio is outside the reference range. In other words, the result of the determination by this function 1203 corresponds to estimation that the measured sample 5 is not consistent with smoking effects on the test subject U1.

Determination Condition 4: A function 1204 of determining whether or not the sample is consistent with drinking effects: This function 1204 references, from the questionnaire information 1704, information on a situation such as a drinking status of the test subject U1 corresponding to the target sample 5. This function 1204 obtains, as the result of measuring the sample 5, a component amount ratio in alcohol as the target component substance from the mass measurement data 1701. This function 1204 references, from the reference range information 1705, a reference range of the component amount ratio in the sample 5, which is alcohol, as the target component substance. This function 1204 compares the component amount ratio in alcohol as the target component substance in the measurement result to the reference range to be referenced, and determines the sample to be inappropriate when the component amount ratio is outside the reference range. In other words, the result of the determination by this function 1204 corresponds to estimation that the measured sample 5 is not consistent with drinking effects on the test subject U1.

Effects of tobacco and alcohol intake appears as related substances contained in some body fluids. Therefore, by examining component amounts of a tobacco-related substance and component amounts of an alcohol-related substance, the function 1203 and the function 1204 can estimate whether or not the sample 5 is consistent with the smoking and drinking statues of the test subject of concern and whether 4 not there is a possibility that the sample 5 has been collected from a person with smoking and drinking statuses different from those of the person of concern.

### Component Amounts of Target Component Substance

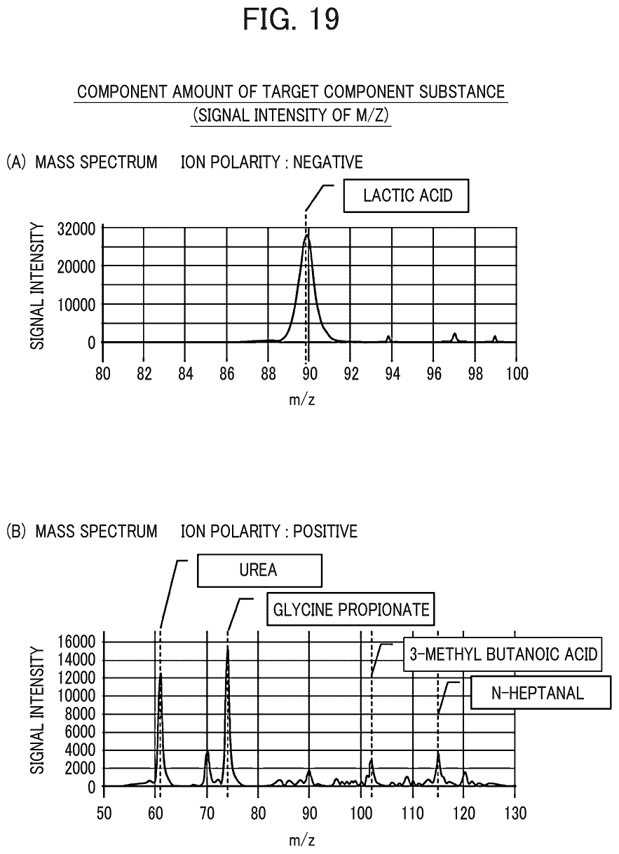

FIG. 19 illustrate an example of a signal intensity of a mass spectrum versus the mass-to-charge ratio (m/z) as component amounts of the target component substance. In the example in each of FIG. 19, a result of detecting human-derived specific components contained in a sample obtained by collecting a skin tissue as a human body fluid. For example, the function 1202 for the determination condition 2 can use each of such human-derived components as the target substance. The mass spectrum in FIG. 19(A) indicates a m/z range of one of the components having a negative ion polarity, while the mass spectrum in FIG. 19(B) indicates m/z ranges of some of the components each having a positive ion polarity. In FIG. 19(A), a lactic acid is detected as a peak of the signal intensity around m/z 90. In FIG. 19(B), urea, glycine propionate 3-methylbutane, and N-heptanal were respectively detected around m/z 60, m/z 74, m/z 102, and m/z 115.

### Impurities

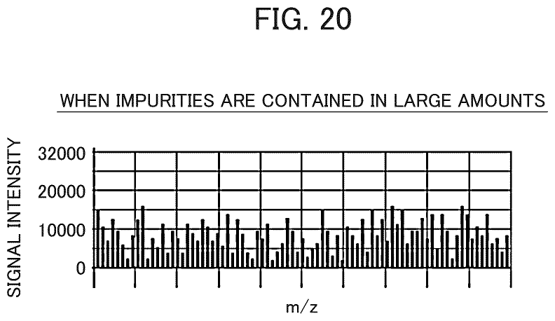

FIG. 20 illustrates a case where, in the mass spectrum, foreign substances appear all over in a m/z range. Such a case is referred to as impurities. The impurities are substances other than human body fluids. In a case where the impurities are thus detected in large amounts, even if there is a peak at a m/z location of the target component substance, it is doubtful whether the sample has been collected from a human being. Accordingly, the function 1202 for the determination condition 2 also takes the amounts of the impurities into consideration. The function 1202 counts all the impurities where a signal intensity of a certain level or more is observed, and calculates a total value of the impurity amounts. When the total value of the impurity amounts exceeds a predetermined value, the function 1202 estimates that the sample has not been collected from a human being.

In the first embodiment, the function 1200 implements all of the foregoing determination conditions 1 to 4, but the determination conditions are not limited thereto, and it may also be possible to use a mode in which at least one of the determination conditions is implemented. In a case of making determination under each of the plurality of determination conditions, determination accuracy can be increased.

### Determination Condition 1

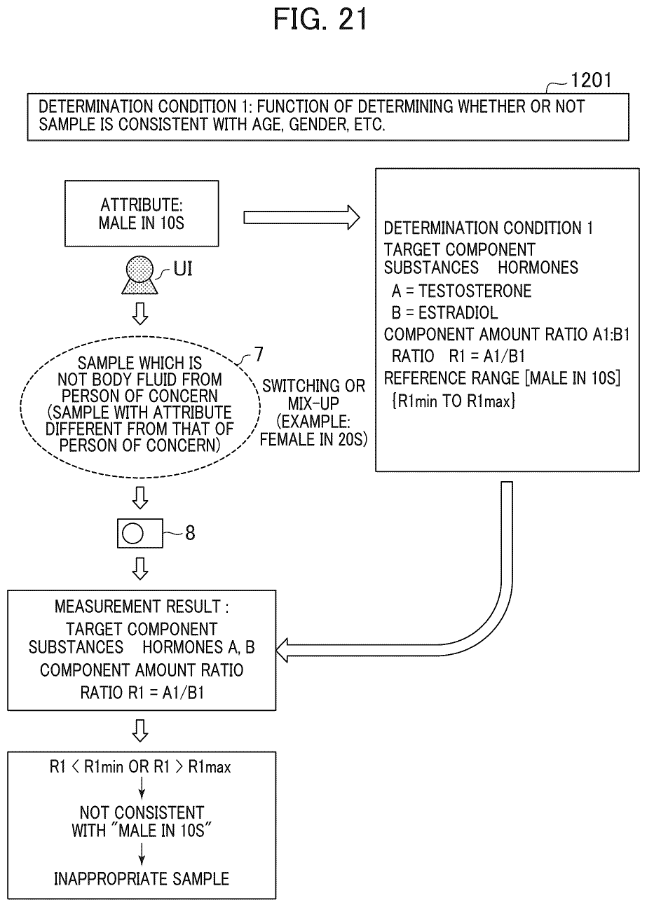

FIG. 21 illustrates a specific example of the determination of whether or not the sample is consistent with the age and gender by the function 1201 for the determination condition 1. In an example of processing under the determination condition 1, a component amount ratio among a plurality of types of hormones, each as the target component substance, is used. Examples of the hormones, each as the target component substance, include testosterone, estradiol, and the like. Depending on the age and the gender, hormone secretion differs, and a hormone component amount ratio differs. For example, a component amount ratio between testosterone and estradiol differs. The m/z of testosterone is 289, while the m/z of estradiol is 272. The function 1201 for the determination condition 1 provides, e.g., a plurality of categories such as a male teenager and a female teenager, and sets respective standard ranges for the individual categories.

For example, it is assumed that testosterone is A, estradiol is B, and a component amount ratio therebetween is A:B. In that case, e.g., in the male teenager category, a reference value of the component amount ratio is assumed to be A1:B1 while, in the female teenager category, a reference value of the component amount ratio is assumed to be A2:B2. Note that the component amount ratio is represented herein by a ratio (A:B) (where colon “:” represents a ratio), but may also be represented by a fraction (R=A/B) (where slash “/” represents division). In addition, the reference value is provided with a range (e.g., a lower limit value and an upper limit value). For example, for the fraction of A1: B1 (R1=A1/B1), the lower limit value is provided as R1ₘᵢₙ, while the upper limit value is provided as R1ₘₐₓ.

It is assumed on the basis of the information in the questionnaire 7 that the test subject U1 is, e.g., a male in his 10s. The function 1201 references a reference range corresponding to the male in his 10s, e.g., {R1ₘᵢₙ to R1ₘₐₓ} . The function 1201 obtains the component amount ration between the hormones A and B in the measurement result, e.g., the fraction R1=A1/B1. The function 1201 compares the fraction R1 of the component amount ratio to the reference range {R1ₘᵢₙ to R1ₘₐₓ}. When the fraction R1 is outside the reference range, i.e., R1 is less than the lower limit value R1ₘᵢₙ (R1<R1ₘᵢₙ) or (OR), when R1 is more than the upper limit value R1ₘₐₓ, (R1>R1ₘₐₓ), a determination result is an inappropriate sample. When the sample 5 submitted from the test subject U1 is a sample collected from another person, e.g., a female in her 20s, the measurement result is not consistent with a characteristic of the hormones of the male in his 10s, and consequently the determination result is an inappropriate sample.

### Determination Condition 2

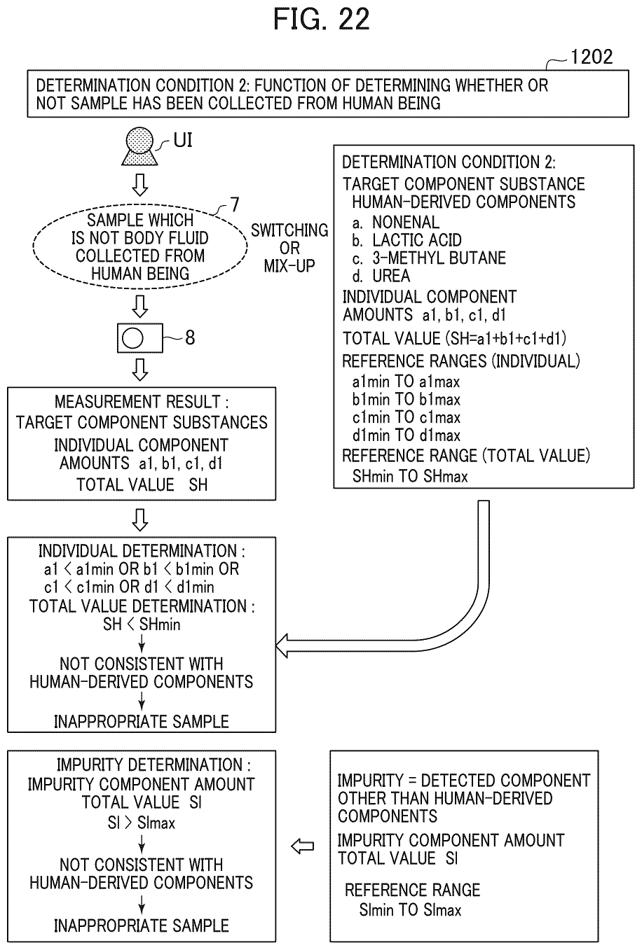

FIG. 22 illustrates a specific example of the determination of whether or not the sample has been collected from a human being by the function 1202 for the determination condition 2. In an example of processing under the determination condition 2, a total value of the component amounts of the human-derived components and a total value of the impurities are used. As the human-derived components each as the target component substance, e.g., nonenal, the lactic acid, 3-methylbutane, urea, or the like as shown in FIG. 19 can be used. The m/z of nonenal is 140, the m/z of the lactic acid is 90, the m/z of 3-methylbutane is 102, and the m/z of urea is 60.

The function 1202 provides a reference range for each of the human-derived component substances as the target component substance or for the total amount of the human-derived components. The function 1202 calculates the component amounts of each of the human-derived component substances and a total value thereof. The function 1202 may compare the component amount of each of the human-derived component substances to the reference range, or may also use the total value of the component amounts of the human-derived component substance to compare the total value of the component amounts to the reference range related to the total value as simple determination.

In this example, as the human-derived components, nonenal, the lactic acid, 3-methylbutane, and urea are assumed to be a substance a, a substance b, a substance c, and a substance d, and the respective component amounts thereof are assumed to be a1, b1, c1, and d1. In addition, a total value of the component amounts of the four substances is assumed to be given by SH=a1+b1+c1+d1. It may also be possible that, for the individual human-derived component substances, the respective reference ranges are provided. For example, for the substance a, as a lower limit value a1ₘᵢₙ and an upper limit value a1ₘₐₓ, a reference range {a1ₘᵢₙ-a1ₘₐₓ} is provided.

In addition, for a reference range related to a total value SH, as a lower limit value SHₘᵢₙ and an upper limit value SHₘₐₓ, a reference range {SHₘᵢₙ-SHₘₐₓ} is provided.

In a case of making determination for each of the individual components, when the component amount a1 of the substance a in the result of measuring the sample 5 is less than the lower limit value a1ₘᵢₙ of the reference range, the sample is determined to be inappropriate. Likewise, when the component amount b1 of the substance b is less than a lower limit value b1ₘᵢₙ, the component amount c1 of the substance c is less than a lower limit value c1ₘᵢₙ, or the component amount d1 of the substance d is less than a lower limit value d1ₘᵢₙ, the sample is determined to be inappropriate. In a case of determining the sample on the basis of a total value, when, e.g., the total value SH of the component amounts of the four substances in the measurement result of the sample 5 is less than the lower limit value SHₘᵢₙ of the reference range, the sample is determined to be inappropriate.

The function 1202 may further make the determination by using the component amounts of the impurities in the result of measuring the sample 5. The impurities are substances other than the human-derived components. In a case of determining the impurities, the function 1202 calculates, e.g., the total value of the component amounts of the impurities. It is assumed that the total value of the component amounts of the impurities is SI. The function 1202 provides, as the reference range related to the total value of the component amounts of the impurities, e.g., a lower limit value SIₘᵢₙ and an upper limit value SIₘₐₓ. The function 1202 determines the sample to be inappropriate when the total value SI of the component amounts of the impurities in the result of measuring the sample 5 exceeds, e.g., the upper limit value SIₘₐₓ.

When the sample 5 submitted from the test subject U1 is not the body fluid collected from the person of concern, who is a human being, but is an object collected from a non-living being as a result of switching or the like, the sample is determined to be inappropriate, since a measurement result is not consistent with the human-derived components.

It is also possible that the function 1202 for the determination condition 2 combines the attributes, such as the age and the gender, under the determination condition 1. For example, the function 1202 may refer to the age, the gender, and the like of the sample on the basis of the questionnaire information, and may reference the reference range of the human-derived component substance which differs according to the categories of the age and the gender. For example, in a case of a middle-aged male, reference or the like is made to a reference range of a specific substance (such as, e.g., nonenal) corresponding to the middle-aged male.

### Determination Condition 3

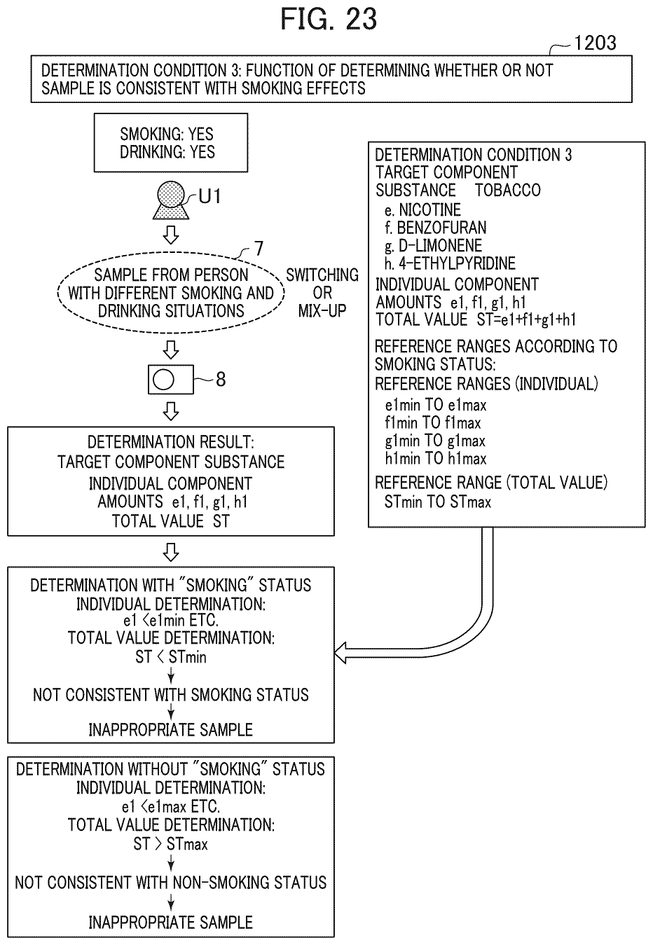

FIG. 23 illustrates a specific example of determination of whether or not the sample is consistent with the smoking effects made by the function 1203 for the determination condition 3. On the basis of the questionnaire information, it is determined that the test subject U1 has, e.g., smoking and drinking habits. Under the determination condition 3, it is assumed that, as the target component substances related to tobacco, e.g., nicotine, benzofuran, D-limonene, and 4-ethylpyridine are handled, these four substances are substances e, f, g, and h, the respective component amounts thereof are e1, f1, g1, and h1, and a total amount thereof is given by ST=e1+f1+g1+h1. The function 1203 provides respective reference ranges for the individual substances. For example, for the substance e, a reference range such as {e1ₘᵢₙ-e1ₘₐₓ} is provided. In addition, a reference range related to the total value of the component amounts of the four substances, such as {STₘᵢₙ-STₘₐₓ}, is provided. These reference ranges are provided as, e.g., values differing depending on the smoking status.

For example, since the test subject U1 has the smoking habit, the function 1203 references the reference range corresponding to the smoking status. In a case of individual determination, when the component amount of the target component substance in the result of measuring the sample 5, e.g., e1 is less than the lower limit value e1ₘᵢₙ, the sample is determined to be inappropriate. In a case of total value determination, when the total value ST of the component amounts of the four target component substances is less than, e.g., the lower limit value STₘᵢₙ, the sample is determined to be inappropriate.

In an example of another test subject U1, when the test subject U1 has no smoking habit, a reference range corresponding to a non-smoking status is referenced. In the case of the individual determination, when the component amount of the target component substance in the measurement result, e.g., e1 is exceeding the upper limit value e1ₘₐₓ, the sample is determined to be inappropriate. In a case of the total value determination, when the total value ST of the component amounts of the four target component substances is less than, e.g., the upper limit value STₘₐₓ, the sample is determined to be inappropriate.

In a case where the sample 5 submitted from the test subject U1 is the body fluid collected from a person with a smoking status different from that of the person of concern as a result of switching or the like, a measurement result is not consistent with the smoking status (e.g., with smoking), and therefore the sample is determined to be inappropriate. To the function 1204 of the determination condition 4 also, the same processing example as that under the determination condition 3 described above is applicable.

### Component Amount and Reference Range

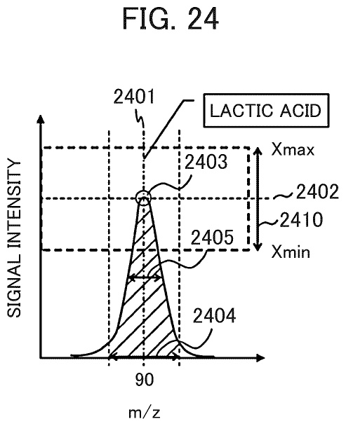

FIG. 24 illustrates, as a supplement, an example of a comparison between the component amounts in the foregoing measurement result and the reference ranges. The present example assumes a case where the target component substance is the lactic acid in FIG. 19(A). As the component amount of the lactic acid, the signal intensity versus m/z is used. In the case of the lactic acid, the signal intensity has a peak value of the signal intensity around m/z 90. 2401 denotes a mass axis, which corresponds to a m/z peak value. 2402 denotes a signal intensity peak value. 2403 denotes a peak portion of a waveform. 2404 denotes a mass width. 2405 denotes an intermediate mass width. In the embodiment, as the component amount of the target component substance, e.g., the signal intensity peak value 2402 is used. The component amount is not limited thereto and, as the component amount, any of various parameter values obtained from the waveform of the mass spectrum (such as, e.g., an intermediate mass width, a resolution, or an integrated value) may also be used.

In relation to the signal intensity peak value 2402 as the component amount, as the reference range, a reference range 2410 from a lower limit value Xₘᵢₙ to a lower limit value Xₘₐₓ is provided. Note that the reference range may also be expressed in another manner as a range having a vertical width around a reference value.

What parameter values are to be used to calculate the component amounts and the reference range can be set as control values. In a control program of the function 1200, the values can be set.

### Example of Screen Displayed on PC of Test Apparatus

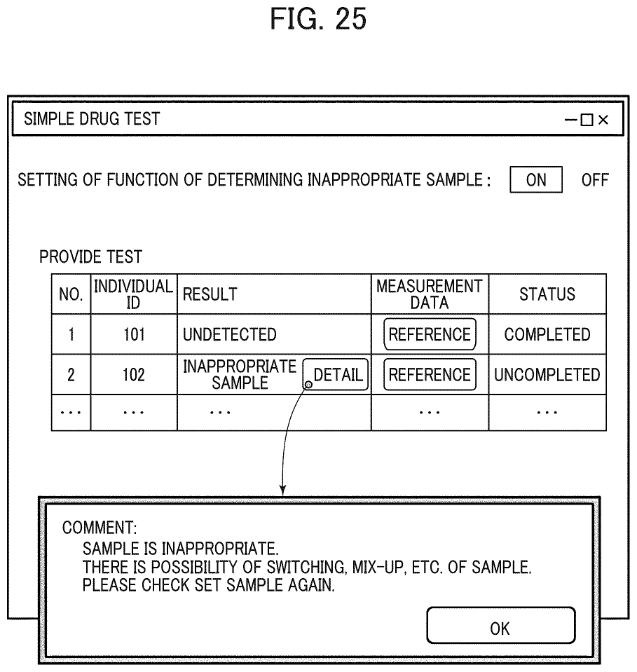

FIG. 25 illustrates an example of a screen displayed on the PC 403 of the test apparatus 1. The present example is an example in which, during testing work by the test provider U3, a result of the determination by the function 1200 of determining the inappropriate sample or the like is displayed as work support information on the screen of the PC 403. The test provider U3 sequentially sets the sample in the test apparatus 1 and performs the testing operation according to the support information on the screen. During the test, when the sample is determined/detected by the function 1200 of determining the inappropriate sample to be inappropriate, an “alert” is output as the “inappropriate sample” to the screen. Alternatively, by pressing a detail button for the “inappropriate sample”, a detailed comment may also be displayed. For example, the comment is a message such that “SAMPLE IS INAPPROPRIATE. THERE IS POSSIBILITY OF SWITCHING, MIX-UP, ETC. OF SAMPLE. PLEASE CHECK SET SAMPLE AGAIN”. The alert output is not limited to display only, and may also be combined with an audio output, a message notification, or the like. In receipt of the alert output “INAPPROPRIATE SAMPLE” on the screen, the test provider U3 confirms the sample 5 (sample holder 6) set in the set apparatus 1.

In addition, the application of the function 1200 of determining the inappropriate sample can be turned ON/OFF on the screen. For example, when the sample 5 is collected directly from the test subject U1, and the test apparatus 1 performs the test on the spot by using the sample 5, the possibility of switching or the like is low, and therefore the test can be performed by turning OFF the function 1200. In this case, there is no need to reference/acquire the questionnaire information. Step S103 in FIG. 10 is skipped.

Note that, according to a type of a test such as an illegal drug test or a doping test, the targe component substance differs, and data (target component substance data 1702 in FIG. 17) is set in advance in the test apparatus 1. As long as the type of the test is specified on the screen of the PC 403, a determination related to the target component substance corresponding to the type of the test is possible, though not shown.

### Modification

In the first embodiment, the mode has been described in which, to determine the inappropriate sample, the function 1200 references the attribute information, such as the age of the test subject U1, from the questionnaire input information (i.e., test subject information). In a modification, it may also be possible to determine the inappropriate sample by using only the mass measurement data without using the questionnaire input information (i.e., test subject information).

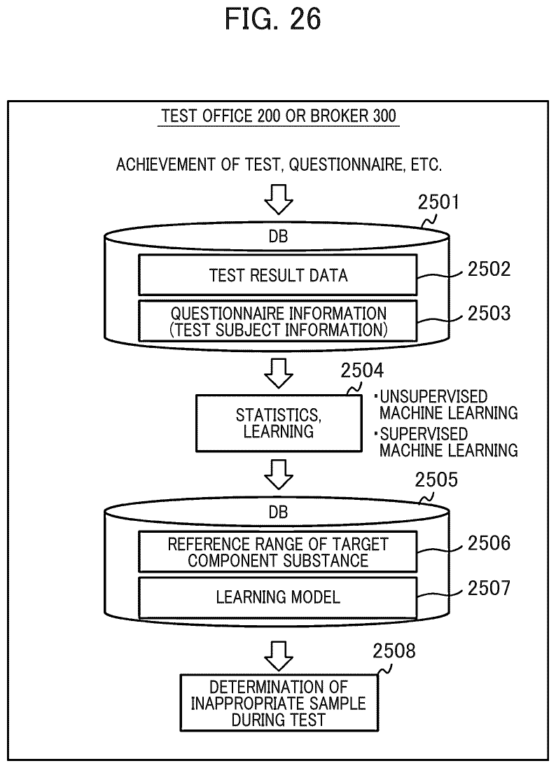

In another modification, the reference ranges related to the component amounts are not limited to being static set values, and may also be dynamically updated set values. FIG. 26 illustrates an example of generation of the reference ranges in the modification. In this modification, the test system, e.g., the broker 300 or the test office 200 stores information on achievements, such as a large number of previous tests and questionnaires, in a DB 2501. In the DB 2501, test result data 2502 including the mass measurement data and questionnaire information 2503 is stored. On the basis of the data in the DB 2501, the test system generates/updates the reference ranges of the component amounts of the target component substances by statistics or learning 2504. In a DB 2505, a reference range 2506 and a learning model 2507 is stored. In a case of using learning, using, e.g., unsupervised machine learning or supervised machine learning, the learning model 2507 is trained. In the learning, e.g., the mass measurement data and the questionnaire information (i.e., test subject information) is input, it is output whether or not the sample is inappropriate, and parameter values of the learning model 2507 are adjusted.

During the test in the test apparatus 1, when making the determination, the function 1200 may also reference the reference range 2506 obtained by previous statistics and make the determination. When there is the trained learning model 2507, by inputting the mass measurement data and the questionnaire information to the learning model 2507, it is possible to output a result of the determination of whether or not the sample is inappropriate as a result of deduction by

### Effects of Embodiments

As described above, according to the test method in the first embodiment or the like, it is possible to determine/detect/output a possibility of intentional switching or falsification, unintentional mix-up or misidentification, or the like as the inappropriate sample, which contributes to optimization and efficiency of the test. According to the first embodiment, when the simple test service is to be provided to a customer by mail, it is possible to alert-output a possibility that a sample is an inappropriate sample which is not a body liquid collected from the person of concern.

In the first embodiment, the test apparatus 1 is provided at the location 2 of the test office 200, which is remote from the location 1 of the customer office 100, and the test office 200 can provide the simple drug test to the customer offices 100 for a plurality of the customers. The test apparatus 1 is used by turning ON the function 1200 of determining the inappropriate sample for each of the samples 5 submitted from the customers. The test apparatus 1 is not limited thereto and, when placed at the location 1 of the customer office 100, the test apparatus 1 may also be used by turning OFF the function 1200.

In the first embodiment, the case has been described in which, as the system that provides the simple test service to the customer office 100 for the customer, the system of the broker 300 and the system of the test office 200 cooperate to provide the simple test service, but the system that provides the simple test service is not limited thereto. It may also be possible that the system of the broker 300 and the system of the test office 200 are configured as an integrated system. It may also be possible that a computer other than the test apparatus 1 is provided with the function 1200 of determining the inappropriate sample.

In the modification, it may also be possible that the server 30 is provided with the function 1200 of determining the inappropriate sample. In that case, the server 30 acquires the measurement result data or the like from the test apparatus 1 by communication. The server 30 determines whether or not the sample is inappropriate on the basis of the questionnaire input information and the measurement result data or the like, and outputs an alert or a report indicating that the sample is inappropriate to the customer office 100 or the test office 200 when determining the sample to be inappropriate.

For a possibility that the sample is from someone other than the person of concern, the function 1200 in the first embodiment provides a binary determination result indicating Yes or No, but the determination result is not limited thereto, and it may also be possible to provide a multi-value determination result. For example, in a comparison between the component amount and the reference range, by providing thresholds at multiple stages, it is possible to represent the determination result with levels, degrees, or the like of three or more values. For example, the determination result may also be expressed such that “There is a medium possibility that the sample is from someone other than the person of concern” or “there is an 80% possibility that the sample is from someone other than the person of concern”.

It is assumed that the function 1200 in the first embodiment implements and uses four determination conditions as in FIG. 18, but the function 1200 is not limited thereto, and it may also be possible to implement and use at least one determination condition. In a case of only a given determination condition, a determination result is that the sample is inappropriate when the sample meets the determination condition. When a plurality of determination conditions are compositely used, one method is that, when the sample corresponds to any one of the determination conditions, the determination result is that the sample is inappropriate. In another method, it may also be possible to further make a comprehensive determination from determination results under the plurality of determination conditions. For example, it may also be possible to make the comprehensive determination resulting from the determination results under the four determination conditions described above by a logic operation or another calculation. By way of example, when the respective determination results under the individual determination conditions have value each representing a degree of possibility, it may also be possible to obtain a comprehensive index value or evaluation value by performing addition or multiplication of the values indicating the four determination results.

On the sample collected from a surface or human skin or a skin tissue, effects of tobacco and alcohol are reflected. In addition, on the surface of human skin, there is skin gas generated from a person, and a state of the skin gas is related to physical/physiological states of the person. Effects of these substances can be taken into consideration as the component amounts in the result of measuring the sample, and it is possible to determine whether or not the sample is inappropriate on the basis of whether or not there is consistency with a characteristic of the test subject U1.

In another modification, information such as a current medical history of the test subject U1 may also be used to determine the inappropriate sample on the basis of the questionnaire information. For example, in a case of diabetes, acetone can be listed as one of skin gas substances that are closely related to diabetes. When acetone is treated as a target component substance and an amount of acetone component is, e.g., less than a lower limit, it is possible to estimate a possibility that the sample is not from the person of concern.

While the embodiments of the present disclosure have been specially described, the present disclosure is not limited to the embodiments described above and can variously be modified within the scope not departing from the gist thereof. Except for essential components, the components of each of the embodiments can be subjected to addition, deletion, replacement, or the like. Unless otherwise particularly limited, each component may include a single component or a plurality of components. Modes in which the embodiments and modifications are combined are also possible.

### LIST OF REFERENCE SIGNS

1: test apparatus, 5: sample, 6: sample holder, 7: questionnaire, 8: report, 10: work terminal, 20: management terminal, 30: server, 31: GUI (Web page), 100: customer office, 200: test office, 300: broker, 900: communication network, 1200: function of determining the inappropriate sample, U1: test subject, U2: worker, U3: test provider

1. A body fluid analysis method of using a mass spectrometer serving as a test apparatus to analyze a body fluid, which is a sample collected from a test subject,
the mass spectrometer being an apparatus having a function of introducing the sample and measuring and analyzing a component amount of a target component substance on the basis of mass spectrometry,
the body fluid analysis method comprising, as steps to be performed by the mass spectrometer:
a determination step of determining, as a result of the measurement of the sample by the mass spectrometer, a possibility of switching or mix-up of the sample to be an inappropriate sample on the basis of the component amount of the target component substance; and
an output step of outputting a result of the determination when the sample is determined to be inappropriate.

2. The body fluid analysis method according to claim 1, further comprising the steps of:
requesting a detailed examination when a location where the sample is collected from the test subject is different from the location where the test apparatus for testing the sample is placed and when the component amount of a target substance to be tested as the target component substance is equal to or more than a reference value on the basis of a result of the measurement; and
requesting, when the sample is determined to be inappropriate, re-testing of the newly collected sample.

3. The body fluid analysis method according to claim 1,
wherein the determination step includes determining the sample to be inappropriate when a ratio among component amounts of a plurality of specific substances according to the test subject, each as the target component substance of the sample, is outside a reference range related to the ratio.

4. The body fluid analysis method according to claim 3,
wherein the plurality of specific substances according to the test subject are a plurality of specific substances in accordance with attributes of the test subject including an age and a gender.

5. The body fluid analysis method according to claim 1,
wherein the determination step includes determining the sample to be inappropriate when a total value of component amounts of a plurality of specific substances contained in the body fluid collected from a person, each as the target component substance of the sample, is outside a reference range related to the total value.

6. The body fluid analysis method according to claim 1,
wherein the determination step includes determining the sample to be inappropriate when a total value of component amounts of impurities other than a plurality of specific substances contained in the body fluid collected from a person, each as the target component substance of the sample, is outside a reference range related to the total value.

7. The body fluid analysis method according to claim 1,
wherein the determination step includes determining the sample to be inappropriate when a component amount of a specific substance related to smoking, as the target component substance of the sample, is outside a reference range related to the component amount correspondingly to a smoking status according to the test subject.

8. The body fluid analysis method according to claim 1,
wherein the determination step includes determining the sample to be inappropriate when a component amount of a specific substance related to drinking alcohol, as the target component substance of the sample, is outside a reference range related to the component amount correspondingly to an alcohol drinking status according to the test subject.

9. The body fluid analysis method according to claim 1, further comprising the step of:
acquiring, with acquisition of the sample, information from a questionnaire containing information on the test subject,
wherein the determination step includes making the determination using the information from the questionnaire.

10. The body fluid analysis method according to claim 1, further comprising the step of:
outputting, to customers including the test subject, a report for notification of a result of testing the sample including a determination result when the sample is determined

11. A body fluid analysis system that uses a mass spectrometer as a test apparatus to analyze a body fluid, which is a sample collected from a test subject,
the mass spectrometer being an apparatus having a function of introducing the sample and measuring and analyzing a component amount of a target component substance on the basis of mass spectrometry,
the mass spectrometer determining, as a result of the measurement of the sample by the mass spectrometer, a possibility of switching or mix-up of the sample to be an inappropriate sample on the basis of the component amount of the target component substance,
the mass spectrometer outputting a result of the determination when the sample is determined to be inappropriate.
